(19) United States
(12) Patent Application Publication
(10) Pub. No.: US 2006/0246437 A1
(43) Pub. Date: Nov. 2, 2006

(54) Genetic susceptibility genes for asthma and atopy and asthma-related and atopic-related phenotypes

(76) Inventors: Margaret A. Pericak-Vance, Chapet Hill, NC (US); Jeffery M. Vance, Chapel Hill, NC (US); Marcy C. Speer, Durham, NC (US); Michael A. Hauser, Durham, NC (US)

Correspondence Address: MYERS BIGEL SIBLEY & SAJOVEC, PO BOX 37428, RALEIGH, NC, 27627, US

(21) Appl. No.: 10/520,695

(22) PCT Filed: Jul. 11, 2003
(86) PCT No.: PCT/US03/21963

§ 371 Date: Nov. 14, 2005

(51) Int. Cl. C12Q 1/68

(52) U.S. Cl. 435/6

(57) ABSTRACT

The present invention is directed to a method for diagnosing a subject as being or having a predisposition to being asthmatic. The present invention is also directed to methods of detecting whether a subject may be atopic by screening for genetic risk factors.

### CROSS REFERENCE TO RELATED APPLICATION

The present application claims priority to U.S. Provisional Application No. 60/395,439, filed Jul. 12, 2002 and U.S. Provisional Application No. 60/428,875, filed Nov. 25, 2002, the disclosures of which are incorporated herein by reference in their entireties.

### FIELD OF THE INVENTION

This invention concerns methods of screening for asthma and/or atopy by the screening of genetic risk factors.

### BACKGROUND OF THE INVENTION

Asthma is a common, complex disorder with both genetic and environmental components. Asthma is often characterized by inflammation of airways, wheeze, cough and bronchial hyper-reactivity to both chemical and physical stimuli. Asthma is also associated with atopy. The clinical features of atopy frequently include total serum high levels of IgE antibodies, antigen-specific IgE responses, and positive skin prick test (SPT) to common airborne allergens. The phenotypically variable expression of atopic asthma appears to be influenced by genetic factors such as population heterogeneity and varying penetrance, and also by non-genetic factors such as uncontrolled environmental factors (i.e. airborne allergens), lifestyle, infections and last, but not least, selection criteria (i.e. age, sex and clinical evaluation). Since allergic asthma is phenotypically variable, the analysis of related but well defined phenotypes might be of importance in identifying genes involved in the different aspects of the disease.

Asthma is a disease which is becoming more prevalent in the modern era and is now the most common disease of childhood. Most asthma found in children and young adults is initiated by an IgE mediated allergy to inhaled allergens such as house dust mite and cat dander. However, not all asthmatics are atopic, and most atopic individuals do not have asthma. Thus, factors in addition to atopy are necessary to induce the disease. Asthma has often been found to be strongly familial, and is due to the interaction between genetic and environmental factors. The genetic factors are thought to be variants of normal genes (“polymorphisms”) which alter their function to predispose to asthma.

Asthma may be identified by recurrent wheeze and intermittent air flow limitation. Asthma often results in mucosal inflammation, wheezing, coughing, sneezing and nasal blockage. Atopy associated with asthma may be diagnosed by (i) a positive skin prick test in response to a common allergen; (ii) detecting the presence of specific serum IgE for allergen; or (iii) by detecting elevation of total serum IgE. Some genetic associations with atopy have been demonstrated. However, the known polymorphisms do not account for all of the genetic factors which predispose to asthma. In particular, asthma is not necessarily an atopic disease. Identification of further genetic polymorphisms linked to asthma will allow the identification of children at risk of asthma before the disease has developed (for example, immediately after birth), with the potential for prevention of disease.

Several genome-wide searches have been performed and candidate chromosomal locations have been reported as being possibly associated with allergic asthma and atopic phenotypes in different ethnic groups. One such study includes a Collaborative Study on the Genetics of Asthma article reports on a genome wide search for linkage of asthma associated phenotypes in regions of chromosomes 5p15, 5q23-31, 6p21-23, 17p11.1-q11.2, 19q13 and 21q21. The Collaborative Study on the Genetics of Asthma (CSGA), A Genome-Wide Search for Asthma Susceptibility Loci in Ethnically Diverse Populations, Nat Genet 15: 389-92 (1997). Additionally the authors note that other studies have reported linkage of bronchial hyper-responsiveness and atopy to chromosomes 5q and 6p.

Another group has provided a review article that discloses that chromosome 5 and 6 demonstrate linkage to asthma related phenotypes. Cookson et al., Genetics of Asthma and Allergic Disease, Human Mol Genetics 9: 2359-64 (2000). This group also has filed a patent application that recites methods for diagnosing or testing a subject as being asthmatic wherein the individual has the presence of an unusual variant form of at least one polymorphic sequence in the MHC region of chromosome 6p. Cookson et al., U.S. Patent Application Publication No.: US2001/0007741. The application also claims a kit for the diagnosis or prognosis of asthma comprising oligonucleotide primers which amplify a polynucleotide sequence in the MHC region of chromosome 6p and contain a polymorphism. This application claims a priority date of April 1997.

Other groups have also reported on the results of a genome-wide search for linkage to traits associated with allergic (atopic) asthma. Daniels, et al., A genome wide search for quantitative trait underlying asthma, _{Nature,} 383: 247-250 (1996). This group found identified six positive linkages with five associated to qualitative traits. Of these, chromosomes 6 and 7 were identified as showing linkage.

Another review article compiles various pieces of evidence that support linkage and associations to the various genetic loci and candidate genes. Hakonarson H, et al., Current Concepts on the Genetics of Asthma, Curr Opin Pediatr 13: 267-77 (2001). The article notes that the roles of genes and genetic variations within the present candidates asthma genes that associate with the asthmatic phenotype remains to be determined.

The Hizawa et al. group also describe a genome-wide screening for genes for genes implicated in atopic diseases. Hizawa N. et al., Genetic Regulation of Dermotaophagoides Pteronyssinus-Specific IgE Responsiveness: A Genome-Wide Multipoint Linkage Analysis in Families Recruited Through 2 Asthmatic Sibs, J Allergy Clin Immunol 102: 436-442 (1998); Hizawa N. et al., Linkage Analysis of Dermatophagoides Pteronyssinus-Specific IgE Responsiveness with Polymorphic Markers on Chromosome 6p21 (HLA-D region) in Caucasian Families by the Transmission/Disequilibrium Test, J Allergy Clin Immunol 102: 443-8 (1998); and Hizawa N. et al., Genetic Influences on Chromosomes 5q31-q33 and 11q13 on Specific IgE Responsiveness to Common Inhaled Allergens Among African American families, J Allergy Clin Immunol 102: 449-53 (1998). Specifically, chromosomes 5q23-33, 6p21 and 8p23-p21 were singled out as possibly showing linkage.

The Laitinen et al. group was able to describe evidence of linkage in a region of chromosome 19 p13 and 7 p14-p15 for phenotypes of asthma, IgE and a combination thereof. Laitinen et al., Association Study of the Chromosomal Region Containing the FCER2 Gene Suggests It Has a Regulatory Role in Atopic Disorders. Am J Respir Crit Care Med 161: 700-706 (2000); and Laitinen, T., et al., A Susceptibility Locus For Asthma-Related Trails on Chromosome 7 Revealed by Genome-Wide Scan in a Founder Population, Nat Genet 28: 87-91 (2001). These also disclose the satellite markers D19S534, D19S822, D19S884, D19S216, D19S567, D19S120, D19S536, D19S413.

Noguchi et al. discuss the possible linkage on chromosome 5 as a candidate for one of the genes for atopy and asthma. Noguchi E, et al., Mutation Screening of Interferon Regulatory Factor 1 Gene (IRF-1) as a Candidate Gene for Atopy/Asthma, Clin Exp Allergy 30: 1562-7 (2000).

The Ober et al. group discuss mapping genetic traits and identifying genes that are likely to influence asthma. Ober C. et al., Genome-Wide Search for Asthma Susceptibility Loci in a Founder Population, Hum Mol Genet 7: 1393-8 (1998); Ober C. et al., The Genetics of Asthma. Mapping Genes for Complex Traits in Founder Populations, Clin Exp Allergy 28 Suppl 1: 101-5; discussion 108-10 (1998); Ober C. et al., Do Genetics Play a Role in the Pathogenesis of Asthma? Allergy Clin Immunol 101: S417-20 (1998); Ober C, et al., Contributing Factors to the Pathobiology. The Genetics of Asthma, Clin Chest Med 21: 245-61 (2000); Ober C, et al., A Second-Generation Genomewide Screen for Asthma-Susceptibility Al leles in a Founder Population, Am J Hum Genet 67: 1154-62 (2000); and Summerhill E, et al., Beta(2)-Adrenergic Receptor Arg16/Arg 16 Genotype is Associated with Reduced Lung Function, but Not with Asthma, in the Hutterites, Am J Respir Crit Care Med 162: 599-602 (2000). The regions from these articles include chromosomes 5p, 5q, 6p, 6p21, 8p, 9p22, 19q19, and 21q21. The markers disclosed by Ober et al. include D1S468, D1S1597, D1S3669, D1S239, D5S1470, D5S1480, D5S1462, D5S1453, D5S2014, D8S1110, D8S1477, D8S1136, D8S2324, D9S925, D9S922, D9S938, D19S900, D19S540, D19S178, D21S1262, D19S1440, D19S900 and D19S540.

Wjst et al. have demonstrated linkage analysis for asthma by chromosomes 1p36, 1p33, 1p21, 1pter, 5, 6, 6p21-25, 7cen, 7pter, 7p21, 7 p15, 8, 9, 9q13, 9q23, 9q32, 10 and 15q22. Wjst M., et al., “A Genome-Wide Search for Linkage to Asthma”, German Asthma Genetics Group, Genomics 58: 1-8 (1999).

Furthermore, the Xu et al. articles indicate evidence of linkage with the asthma phenotype at 1p32, 5q31-33, 6p21, 7p and 8p23. Xu et al., Major genes regulating total serum immunoglobulin E levels in families with asthma, Am J Hum Genet 67: 1163-73 (2000); and Xu J. et al., Genomewide Screen and Identification of Gene-Gene Interactions for Asthma-Susceptibility Loci in Three U.S. Populations: Collaborative Study on the Genetics of Asthma, Am J Hum Genet 68: 1437-46 (2001). One of the markers disclosed by Xu includes D7S821.

Nevertheless, the genetic basis for asthma and/or atopy is not well understood, and there is a continued need to develop new genetic linkages and markers as well as identify new functional polymorphisms that are associated with asthma and/or atopy. Because asthma and/or atopy share some common clinical and pathological findings, it may be advantageous to locate a gene or genes common to both disorders.

### SUMMARY OF THE INVENTION

The present invention discloses methods of screening a subject for asthma and/or atopy. In a representative embodiment, one method comprises the steps of: detecting the presence or absence of a marker for asthma and/or atopy, and/or a functional polymorphism associated with a gene linked to asthma and/or atopy, with the presence of such a marker and/or functional polymorphism indicating that the subject is afflicted with or at risk of developing asthma and/or atopy. The detecting step may include detecting whether the subject is heterozygous or homozygous for the marker and/or functional polymorphism, with subjects who are at least heterozygous for the functional polymorphism being at increased risk for asthma and/or atopy. The step of detecting the presence or absence of the marker and/or functional polymorphism may include the step of detecting the presence or absence of the marker and/or functional polymorphism in both chromosomes of the subject (i.e., detecting the presence or absence of one or two alleles containing the marker and/or functional polymorphism). More than one copy of a marker or functional polymorphism (i.e., subjects homozygous for the functional polymorphism) may indicate greater risk of asthma and/or atopy as compared to heterozygous subjects.

A further aspect of the present invention is the use of a device, system, apparatus, reagent, marker, instrument, and/or machine to detect a marker, functional polymorphism and/or mutation as described herein in screening a subject for asthma and/or atopy as described herein.

The foregoing and other objects and aspects of the present invention are explained in detail in the drawings herein and the specification set forth below.

### BRIEF DESCRIPTION OF THE DRAWINGS

FIG: 1 depicts markers in chromosome 1 that indicate asthma or atopy using the Genehunter+ algorithm;

FIG. 2 depicts markers in chromosome 2 that indicate asthma or atopy using the Genehunter+ algorithm;

FIG. 3 depicts markers in chromosome 3 that indicate asthma or atopy using the Genehunter+ algorithm;

FIG. 4 depicts markers in chromosome 4 that indicate asthma or atopy using the Genehunter+ algorithm;

FIG. 5 depicts markers in chromosome 5 that indicate asthma or atopy using the Genehunter+ algorithm;

FIG. 6 depicts markers in chromosome 6 that indicate asthma or atopy using the Genehunter+ algorithm;

FIG. 7 depicts markers in chromosome 7 that indicate asthma or atopy using the Genehunter+ algorithm;

FIG. 8 depicts markers in chromosome 8 that indicate asthma or atopy using the Genehunter+ algorithm;

FIG. 9 depicts markers in chromosome 9 that indicate asthma or atopy using the Genehunter+ algorithm;

FIG. 10 depicts markers in chromosome 10 that indicate asthma or atopy using the Genehunter+ algorithm;

FIG. 11 depicts markers in chromosome 11 that indicate asthma or atopy using the Genehunter+ algorithm;

FIG. 12 depicts markers in chromosome 12 that indicate asthma or atopy using the Genehunter+ algorithm;

FIG. 13 depicts markers in chromosome 13 that indicate asthma or atopy using the Genehunter+ algorithm;

FIG. 14 depicts markers in chromosome 14 that indicate asthma or atopy using the Genehunter+ algorithm;

FIG. 15 depicts markers in chromosome 15 that indicate asthma or atopy using the Genehunter+ algorithm;

FIG. 16 depicts markers in chromosome 16 that indicate asthma or atopy using the Genehunter+ algorithm;

FIG. 17 depicts markers in chromosome 17 that indicate asthma or atopy using the Genehunter+ algorithm;

FIG. 18 depicts markers in chromosome 18 that indicate asthma or atopy using the Genehunter+ algorithm;

FIG. 19 depicts markers in chromosome 19 that indicate asthma or atopy using the Genehunter+ algorithm;

FIG. 20 depicts markers in chromosome 20 that indicate asthma or atopy using the Genehunter+ algorithm;

FIG. 21 depicts markers in chromosome 21 that indicate asthma or atopy using the Genehunter+ algorithm;

FIG. 22 depicts markers in chromosome 22 that indicate asthma or atopy using the Genehunter+ algorithm; and

FIG. 23 depicts markers in chromosome 23 that indicate asthma or atopy using the Genehunter+ algorithm.

FIG. 24 depicts markers in Greek subjects for chromosomes 1-23 that indicate asthma or atopy using the Genehunter+ algorithm.

FIG. 25 depicts markers in Caucausian subjects for chromosomes 1-23 that indicate asthma or atopy using the Genehunter+ algorithm.

FIG. 26 depicts markers in European subjects for chromosomes 1-23 that indicate asthma or atopy using the Genehunter+ algorithm.

FIG. 27 depicts markers in Norwegian subjects for chromosomes 1-23 that indicate asthma or atopy using the Genehunter+ algorithm.

### DETAILED DESCRIPTION OF THE EMBODIMENTS

As noted above, the present invention provides a method of screening (e.g., diagnosing, detecting, determining or prognosing) for asthma and/or atopy in a subject. Subjects with which the present invention is concerned are primarily human subjects, including male and female subjects of any age or race.

The term “asthma” as used herein is intended to cover all types of asthma. Asthma is a disease process that is characterized by paradoxical narrowing of the bronchi (lung passageways) often making breathing difficult.

“Atopy” is a heterogenous disorder characterized by prolonged and enhanced immunoglobulin E (IgE) responses to common environmental antigens including pollens, house dust mites, etc. Atopy is what underlies the common diseases of asthma and rhinitis (hay fever).

“Screening” as used herein refers to a procedure used to evaluate a subject for risk of asthma and/or atopy. It is not required that the screening procedure be free of false positives or false negatives, as long as the screening procedure is useful and beneficial in determining which of those individuals within a group or population of individuals are at increased risk of asthma and/or atopy. A screening procedure may be carried out for both prognostic and diagnostic purposes (i.e., prognostic methods and diagnostic methods).

“Prognostic method” refers to methods used to help predict, at least in part, the course of a disease. For example, a screening procedure may be carried out on a subject that has not previously been diagnosed with asthma and/or atopy or does not show substantial disease symptoms. The procedure may allow one to obtain an indication of the future likelihood that the subject will be afflicted with asthma and/or atopy. In addition, a prognostic method may be carried out on a subject previously diagnosed with asthma and/or atopy when it is desired to gain greater insight into how the disease will progress for that particular subject (e.g., the likelihood that a particular patient will respond favorably to a particular drug treatment, or when it is desired to classify or separate asthma and/or atopic patients into distinct and different subpopulations for the purpose of conducting a clinical trial thereon). A prognostic method may also be used to determine whether a person will respond to a particular drug.

“Diagnostic method” as used herein refers to a screening procedure carried out on a subject that has previously been determined to at risk for a particular asthma and/or atopic associated disorder due to the presentation of symptoms or the results of another (typically different) screening test.

“Functional polymorphism” as used herein refers to a change in the base pair sequence of a gene that produces a qualitative or quantitative change in the activity of the protein encoded by that gene (e.g., a change in specificity of activity; a change in level of activity). The presence of a functional polymorphism indicates that the subject is at greater risk of developing a particular disease as compared to the general population. For example, the patient carrying the functional polymorphism may be particularly susceptible to chronic exposure to environmental toxins that contribute to asthma and/or atopy. The term “functional polymorphism” includes mutations, deletions and insertions.

A “present” functional polymorphism as used herein (e.g., one that is indicative of or a risk factor for asthma and/or atopy) refers to the nucleic acid sequence corresponding to the functional polymorphism that is found less frequently in the general population relative to asthma and/or atopy as compared to the alternate nucleic acid sequence or sequences found when such functional polymorphism is said to be “absent”.

An “allele” is any one of a series of two or more different genes that occupy the same position (locus) on a chromosome.

“Mutation” as used herein sometimes refers to a functional polymorphism that occurs in less than one percent of the population, and is strongly correlated to the presence of a gene (i.e., the presence of such mutation indicating a high risk of the subject being afflicted with a disease). However, “mutation” is also used herein to refer to a specific site and type of functional polymorphism, without reference to the degree of risk that particular mutation poses to an individual for a particular disease.

“Linked” as used herein refers to a region of a chromosome that is shared more frequently in family members affected by a particular disease, than expected by chance, thereby indicating that the gene or genes within the linked chromosome region contain or are associated with a marker or functional polymorphism that is correlated to the presence of, or risk of, disease. Once linkage is established association studies (linkage disequilibrium) can be used to narrow the region of interest or to identify the risk conferring gene for asthma and/or atopy.

“Associated with” when used to refer to a marker or functional polymorphism and a particular gene means that the functional polymorphism is either within the indicated gene, or in a different physically adjacent gene on that chromosome. In general, such a physically adjacent gene is on the same chromosome and within three centimorgans of the named gene (i.e., within about three million base pairs of the named gene). Alternatively the marker may be detected within 3, 6, 9, 15, 20, 25, 30, 35, 40, 45, or 50 centimorgans of the named gene.

Markers (e.g., genetic markers such as restriction fragment length polymorphisms and simple sequence length polymorphisms) may be detected directly or indirectly. A marker may, for example, be detected indirectly by detecting or screening for another marker that is tightly linked (e.g., is located within three centimorgans) of that marker. A marker may, for example, be detected directly by a binding site. Alternatively the marker may be detected within 3, 6, 9, 15, 20, 25, 30, 35, 40, 45, or 50 centimorgans of the marker.

The presence of a marker or functional polymorphism associated with a gene linked to asthma and/or atopy indicates that the subject is afflicted with asthma and/or atopy or is at risk of developing asthma and/or atopy. A subject who is “at increased risk of developing asthma and/or atopy” is one who is predisposed to the disease, has genetic susceptibility for the disease or is more likely to develop the disease than subjects in which the detected functional polymorphism is absent.

Suitable subjects include those who have not previously been diagnosed as afflicted with asthma, those who have previously been determined to be at risk of developing asthma, and those who have been initially diagnosed as being afflicted with asthma where confirming information is desired. Thus, it is contemplated that the methods described herein be used in conjunction with other clinical diagnostic information known or described in the art which are used in evaluation of subjects with asthma and/or atopy or suspected to be at risk for developing such disease.

The detecting step may be carried out in accordance with known techniques (See, e.g., U.S. Pat. Nos. 6,027,896 and 5,508,167 to Roses et al.), such as by collecting a biological sample containing DNA or RNA from the subject, and then determining the presence or absence of DNA or RNA encoding or indicative of the functional polymorphism in the biological sample. Any biological sample which contains the DNA or RNA of that subject may be employed, including tissue samples and blood samples, with blood cells being a particularly convenient source.

Determining the presence or absence of DNA or RNA encoding a particular functional polymorphism may be carried out with an oligonucleotide probe labeled with a suitable detectable group, and/or by means of an amplification reaction such as a polymerase chain reaction or ligase chain reaction (the product of which amplification reaction may then be detected with a labeled oligonucleotide probe or a number of other techniques). (The term oligonucleotide also referred to simply as “nucleotide, herein) such as DNA and RNA, oligosaccharides, polylipids, polyesters, polyamides, polyurethanes, polyureas, polyethers, poly (phosphorus derivatives) such as phosphates, phosphonates, phosphoramides, phosphonamides, phosphites, phosphinamides, etc., poly (sulfur derivatives) such as sulfones, sulfonates, sulfites, sulfonamides, sulfenamides, etc., where for the phosphorous and sulfur derivatives the indicated heteroatom for the most part will be bonded to C, H, N, O or S, and combinations thereof.) Further, the detecting step may include the step of detecting whether the subject is heterozygous or homozygous for the particular functional polymorphism. Numerous methods of synthesizing or applying such probe molecules on solid supports (where the probe molecule may be either covalently or non-covalently bound to the solid support) are known, and such probe molecules can be made in accordance with procedures known to those skilled in the art. See, e.g., U.S. Pat. No. 5,565,324 to Still et al., U.S. Pat. No. 5,284,514 to Ellman et al., U.S. Pat. No. 5,445,934 to Fodor et al. (the disclosures of all United States patents cited herein are to be incorporated herein by reference in their entirety). Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. Patent references cited herein be incorporated herein by reference).

Amplification of a selected, or target, nucleic acid sequence may be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the QB replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all allelic types, the types can be distinguished by hybridization with an allelic specific probe, by restriction endonuclease digestion, by electrophoresis on denaturing gradient gels, or other techniques.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to asthma and/or atopy or from sequences which can be generated from such genes in accordance with standard techniques.

Ligase chain reaction (LCR) is also carried out in accordance with known techniques. See, e.g., R. Weiss, Science 254, 1292 (1991). In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to hemochromatosis or from sequences which can be generated from such genes in accordance with standard techniques.

It will be appreciated that the detecting steps described herein may be carried out directly or indirectly. Other means of indirectly determining allelic type include measuring polymorphic markers that are linked to the particular functional polymorphism, as has been demonstrated for the VNTR (variable number tandem repeats).

Molecular biology comprises a wide variety of techniques for the analysis of nucleic acid and protein sequences. Many of these techniques and procedures form the basis of clinical diagnostic assays and tests. These techniques include nucleic acid hybridization analysis, restriction enzyme analysis, genetic sequence analysis, and the separation and purification of nucleic acids and proteins (See, e.g., J. Sambrook, E. F. Fritsch, and T. Maniatis, Molecular Cloning: A Laboratory Manual, 2 Ed., Cold spring Harbor Laboratory Press, Cold Spring Harbor, N.Y., 1989).

Most of these techniques involve carrying out numerous operations (e.g., pipetting, centrifugation, and electrophoresis) on a large number of samples. They are often complex and time consuming, and generally require a high degree of accuracy. Many a technique is limited in its application by a lack of sensitivity, specificity, or reproducibility.

For example, the complete process for carrying out a DNA hybridization analysis for a genetic or infectious disease is very involved. Broadly speaking, the complete process may be divided into a number of steps and sub-steps. In the case of genetic disease diagnosis, the first step involves obtaining the sample (e.g., saliva, blood or tissue). Depending on the type of sample, various pre-treatments would be carried out. The second step involves disrupting or lysing the cells which releases the crude DNA material along with other cellular constituents.

Generally, several sub-steps are necessary to remove cell debris and to further purify the DNA from the crude sample. At this point several options exist for further processing and analysis. One option involves denaturing the DNA and carrying out a direct hybridization analysis in one of many formats (dot blot, microbead, microplate, etc.). A second option, called Southern blot hybridization, involves cleaving the DNA with restriction enzymes, separating the DNA fragments on an electrophoretic gel, blotting the DNA to a membrane filter, and then hybridizing the blot with specific DNA probe sequences. This procedure effectively reduces the complexity of the genomic DNA sample, and thereby helps to improve the hybridization specificity and sensitivity. Unfortunately, this procedure is long and arduous. A third option is to carry out an amplification procedure such as the polymerase chain reaction (PCR) or the strand displacement amplification (SDA) method. These procedures amplify (increase) the number of target DNA sequences relative to non-target sequences. Amplification of target DNA helps to overcome problems related to complexity and sensitivity in genomic DNA analysis. After these sample preparation and DNA processing steps, the actual hybridization reaction is performed. Finally, detection and data analysis convert the hybridization event into an analytical result.

Nucleic acid hybridization analysis generally involves the detection of a very small number of specific target nucleic acids (DNA or RNA) with an excess of probe DNA, among a relatively large amount of complex non-target nucleic acids. A reduction in the complexity of the nucleic acid in a sample is helpful to the detection of low copy numbers (i.e. 10,000 to 100,000) of nucleic acid targets. DNA complexity reduction is achieved to some degree by amplification of target nucleic acid sequences. (See, M. A. Innis et al., PCR Protocols: A Guide to Methods and Applications, Academic Press, 1990, Spargo et al., 1996, Molecular & Cellular Probes, in regard to SDA amplification). This is because amplification of target nucleic acids results in an enormous number of target nucleic acid sequences relative to non-target sequences thereby improving the subsequent target hybridization step.

The actual hybridization reaction represents one of the most important and central steps in the whole process. The hybridization step involves placing the prepared DNA sample in contact with a specific reporter probe at set optimal conditions for hybridization to occur between the target DNA sequence and probe.

Hybridization may be performed in any one of a number of formats. For example, multiple sample nucleic acid hybridization analysis has been conducted in a variety of filter and solid support formats (See Beltz et al., Methods in Enzymology, Vol. 100, Part et al., Eds., Academic Press, New York, Chapter 19, pp. 266-308, 1985). One format, the so-called “dot blot” hybridization, involves the non-covalent attachment of target DNAs to a filter followed by the subsequent hybridization to a radioisotope labeled probe(s). “Dot blot” hybridization gained wide-spread use over the past two decades during which time many versions were developed (see Anderson and Young, in Nucleic Acid Hybridization—A Practical Approach, Hames and Higgins, Eds., IRL Press, Washington, D.C. Chapter 4, pp. 73-111, 1985). For example, the dot blot method has been developed for multiple analyses of genomic mutations (EPA 0228075 to Nanibhushan et al.) and for the detection of overlapping clones and the construction of genomic maps (U.S. Pat. No. 5,219,726 to Evans).

New techniques are being developed for carrying out multiple sample nucleic acid hybridization analysis on micro-formatted multiplex or matrix devices (e.g., DNA chips) (see M. Barinaga, 253 Science, pp. 1489, 1991; W. Bains, 10 Bio/Technology, pp. 757-758, 1992). These methods usually attach specific DNA sequences to very small specific areas of a solid support, such as micro-wells of a DNA chip. These hybridization formats are micro-scale versions of the conventional “dot blot” and “sandwich” hybridization systems.

The micro-formatted hybridization can be used to carry out “sequencing by hybridization” (SBH) (see M. Barinaga, 253 Science, pp. 1489, 1991; W. Bains, 10 Bio/Technology, pp. 757-758, 1992). SBH makes use of all possible n-nucleotide oligomers (n-mers) to identify n-mers in an unknown DNA sample, which are subsequently aligned by algorithm analysis to produce the DNA sequence (See, Drmanac U.S. Pat. No. 5,202,231).

There are two formats for carrying out SBH. The first format involves creating an array of all possible n-mers on a support, which is then hybridized with the target sequence. The second format involves attaching the target sequence to a support, which is sequentially probed with all possible n-mers. Both formats have the fundamental problems of direct probe hybridizations and additional difficulties related to multiplex hybridizations. Southern, (United Kingdom Patent Application GB 8810400, 1988; E. M. Southern et al., 13 Genomics 1008, 1992), proposed using the first format to analyze or sequence DNA. Southern identified a known single point mutation using PCR amplified genomic DNA. Southern also described a method for synthesizing an array of oligonucleotides on a solid support for SBH. However, Southern did not address how to achieve optimal stringency conditions for each oligonucleotide on an array. Drmanac et al., (260 Science 1649-1652, 1993), used the second format to sequence several short (116 bp) DNA sequences. Target DNAs were attached to membrane supports (“dot blot” format). Each filter was sequentially hybridized with 272 labeled 10-mer and 11-mer oligonucleotides. Wide ranges of stringency conditions were used to achieve specific hybridization for each n-mer probe. Washing times varied from 5 minutes to overnight using temperatures from 0° C. to 16° C. Most probes required 3 hours of washing at 16° C. The filters had to be exposed from 2 to 18 hours in order to detect hybridization signals. The overall false positive hybridization rate was 5% in spite of the simple target sequences, the reduced set of oligomer probes, and the use of the most stringent conditions available.

Currently, a variety of methods are available for detection and analysis of the hybridization events. Depending on the reporter group (fluorophore, enzyme, radioisotope, etc.) used to label the DNA probe, detection and analysis are carried out fluorimetrically, colorimetrically, or by autoradiography. By observing and measuring emitted radiation, such as fluorescent radiation or particle emission, information may be obtained about the hybridization events. Even when detection methods have very high intrinsic sensitivity, detection of hybridization events is difficult because of the background presence of non-specifically bound materials. Thus, detection of hybridization events is dependent upon how specific and sensitive hybridization can be made. Concerning genetic analysis, several methods have been developed that have attempted to increase specificity and sensitivity.

One form of genetic analysis is analysis centered on elucidation of single nucleic acid polymorphisms or (“SNPs”). Factors favoring the usage of SNPs are their high abundance in the human genome (especially compared to short tandem repeats, (STRs)), their frequent location within coding or regulatory regions of genes (which can affect protein structure or expression levels), and their stability when passed from one generation to the next (Landegren et al., Genome Research, Vol. 8, pp. 769-776, 1998).

A SNP is defined as any position in the genome that exists in two variants and the most common variant occurs less than 99% of the time. In order to use SNPs as widespread genetic markers, it is crucial to be able to genotype them easily, quickly, accurately, and cost-effectively. It is of great interest to type both large sets of SNPs in order to investigate complex disorders where many loci factor into one disease (Risch and Merikangas, Science, Vol. 273, pp. 1516-1517, 1996), as well as small subsets of SNPs previously demonstrated to be associated with known afflictions.

Numerous techniques are currently available for typing SNPs (for review, see Landegren et al., Genome Research, Vol. 8, pp. 769-776,1998), all of which require target amplification. They include direct sequencing (Carothers et al., BioTechniques, Vol. 7, pp. 494-499, 1989), single-strand conformation polymorphism (Orita et al., Proc. Natl. Acad. Sci. USA, Vol. 86, pp. 2766-2770, 1989), allele-specific amplification (Newton et al., Nucleic Acids Research, Vol. 17, pp. 2503-2516, 1989), restriction digestion (Day and Humphries, Analytical Biochemistry, Vol. 222, pp. 389-395, 1994), and hybridization assays. In their most basic form, hybridization assays function by discriminating short oligonucleotide reporters against matched and mismatched targets. Due to difficulty in determining optimal denaturation conditions, many adaptations to the basic protocol have been developed. These include ligation chain reaction (Wu and Wallace, Gene, Vol. 76, pp. 245-254, 1989) and minisequencing (Syvanen et al., Genomics, Vol. 8, pp. 684-692, 1990). Other enhancements include the use of the 5′-nuclease activity of Taq DNA polymerase (Holland et al., Proc. Natl. Acad. Sci. USA, Vol. 88, pp. 7276-7280, 1991), molecular beacons (Tyagi and Kramer, Nature Biotechnology, Vol. 14, pp. 303-308, 1996), heat denaturation curves (Howell et al., Nature Biotechnology, Vol. 17, pp. 87-88, 1999) and DNA “chips” (Wang et al., Science, Vol. 280, pp. 1077-1082, 1998). While each of these assays are functional, they are limited in their practical application in a clinical setting.

An additional phenomenon discovered to be useful in distinguishing SNPs is the nucleic acid interaction energies or base-stacking energies derived from the hybridization of multiple target specific probes to a single target. (see R. Ornstein et al., “An Optimized Potential Function for the Calculation of Nucleic Acid Interaction Energies”, Biopolymers, Vol. 17, 2341-2360 (1978); J. Norberg and L. Nilsson, Biophysical Journal, Vol. 74, pp. 394-402, (1998); and J. Pieters et al., Nucleic Acids Research, Vol. 17, no. 12, pp. 4551-4565 (1989)). This base-stacking phenomenon is used in a unique format in the current invention to provide highly sensitive Tm differentials allowing the direct detection of SNPs in a nucleic acid sample.

Additional methods have been used to distinguish nucleic acid sequences in related organisms or to sequence DNA. For example, U.S. Pat. No. 5,030,557 by Hogan et al. disclosed that the secondary and tertiary structure of a single stranded target nucleic acid may be affected by binding “helper” oligonucleotides in addition to “probe” oligonucleotides causing a higher Tm to be exhibited between the probe and target nucleic acid. That application however was limited in its approach to using hybridization energies only for altering the secondary and tertiary structure of self-annealing RNA strands which if left unaltered would tend to prevent the probe from hybridizing to the target.

With regard to DNA sequencing, K. Khrapko et al., Federation of European Biochemical Societies Letters, Vol. 256, no. 1,2, pp. 118-122 (1989), for example, disclosed that continuous stacking hybridization resulted in duplex stabilization. Additionally, J. Kieleczawa et al., Science, Vol. 258, pp. 1787-1791 (1992), disclosed the use of contiguous strings of hexamers to prime DNA synthesis wherein the contiguous strings appeared to stabilize priming. Likewise, L. Kotler et al., Proc. Natl. Acad. Sci. USA, Vol. 90, pp. 4241-4245, (1993) disclosed sequence specificity in the priming of DNA sequencing reactions by use of hexamer and pentamer oligonucleotide modules. Further, S. Parinov et al., Nucleic Acids Research, Vol. 24, no. 15, pp. 2998-3004, (1996), disclosed the use of base-stacking oligomers for DNA sequencing in association with passive DNA sequencing microchips. Moreover, G. Yershov et al., Proc. Natl. Acad. Sci. USA, Vol. 93, pp. 4913-4918 (1996), disclosed the application of base-stacking energies in SBH on a passive microchip. In Yershov's example, 10-mer DNA probes were anchored to the surface of the microchip and hybridized to target sequences in conjunction with additional short probes, the combination of which appeared to stabilize binding of the probes. In that format, short segments of nucleic acid sequence could be elucidated for DNA sequencing. Yershov further noted that in their system the destabilizing effect of mismatches was increased using shorter probes (e.g., 5-mers). Use of such short probes in DNA sequencing provided the ability to discern the presence of mismatches along the sequence being probed rather than just a single mismatch at one specified location of the probe/target hybridization complex. Use of longer probes (e.g., 8-mer, 10-mer, and 13-mer oligos) were less functional for such purposes.

It will be appreciated that the detecting steps described herein may be carried out directly or indirectly. Other means of indirectly determining allelic type including measuring polymorphic markers that are linked to the particular functional polymorphism, as has been demonstrated for the VNTR (variable number tandem repeats) and the ApoB alleles (Decorter et al., DNA & Cell Biology 9(6), 461-69 (1990), and collecting and determining differences in the protein encoded by a gene containing a functional variant, as described for ApoE4 in U.S. Pat. Nos. 5,508,167 and 6,027,896 to Roses et al.

Kits for determining if a subject is or was (in the case of deceased subjects) afflicted with or is or was at increased risk of developing asthma and/or atopy will include at least one reagent specific for detecting for the presence or absence of at least one functional polymorphism as described herein and instructions for observing that the subject is or was afflicted with or is or was at increased risk of developing asthma and/or atopy if at least one of the functional polymorphisms is detected. The kit may optionally include one or more nucleic acid probes for the amplification and/or detection of the functional polymorphism by any of the techniques described above, with PCR being currently preferred.

Once identified, short oligonucleotide sequences of 5, 10, 15, 20, 30, 50 or more nucleotides corresponding to the region of the target gene containing the cleavage site may be evaluated for predicted structural features. The suitability of candidate targets may also be evaluated by testing their accessibility to hybridization with complimentary oligonucleotides, using ribonuclease protection assays as are known in the art. DNA encoding enzymatic RNA molecules may be produced in accordance with known techniques. See, e.g., T. Cech et al., U.S. Pat. No. 4,987,071; Keene et al., U.S. Pat. No. 5,559,021; Donson et al., U.S. Pat. No. 5,589,367; Torrence et al., U.S. Pat. No. 5,583,032; Joyce, U.S. Pat. No. 5,580,967; Gold et al. U.S. Pat. No. 5,595,877; Wagner et al., U.S. Pat. No. 5,591,601; and U.S. Pat. No. 5,622,854 (the disclosures of which are to be incorporated herein by reference in their entirety).

The examples which follow are set forth to illustrate the present invention, and are not to be construed as limiting thereof.

Methods and Materials

### EXAMPLES

Eight traits were analyzed: PDA (physician diagnosis of asthma), Atopic asthma, Atopy-broad (at least one SPT 3 or IgE>100 IU), Atopy-cons (at least one SPT 3), Strict asthma (2 of 3 symptoms+BHR), BHR (bronchial hyperresponsiveness), log IgE, and slope of the methacholine dose-response curve.

In the present study, the most interesting results from 389 markers were ranked in approximate order of interest. It is important to point out that the sample sizes for the previously reported genomic screens were relatively small and lacked the power to identify and characterize regions of interest. The Genetics of Asthma International Network (hereinafter “GAIN”) sample is large, and in its entirety of approximately 1100 families, will provide substantial power to more fully characterize regions of interest. The sample typed to date, which represents about 35% of the overall total number of families, is almost three times as large as any previously reported data set.

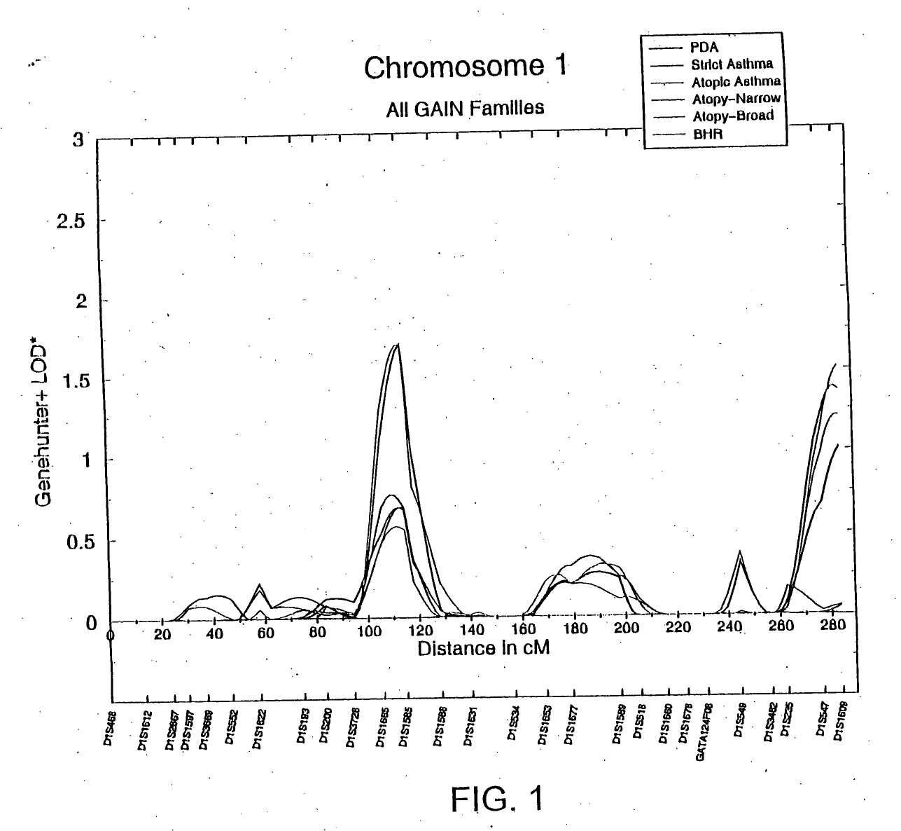

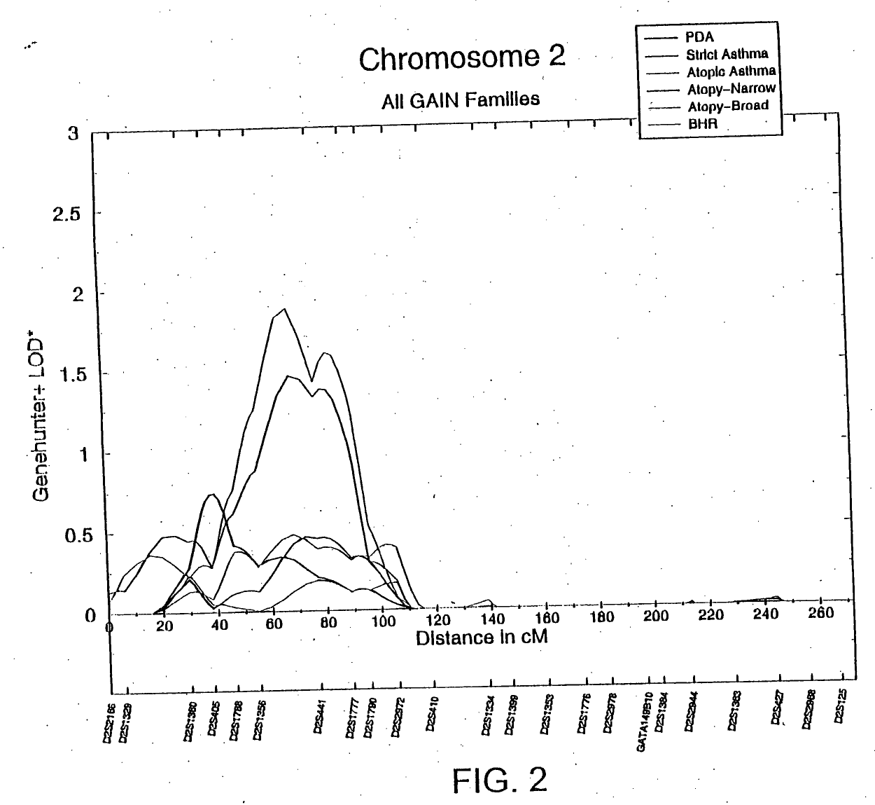

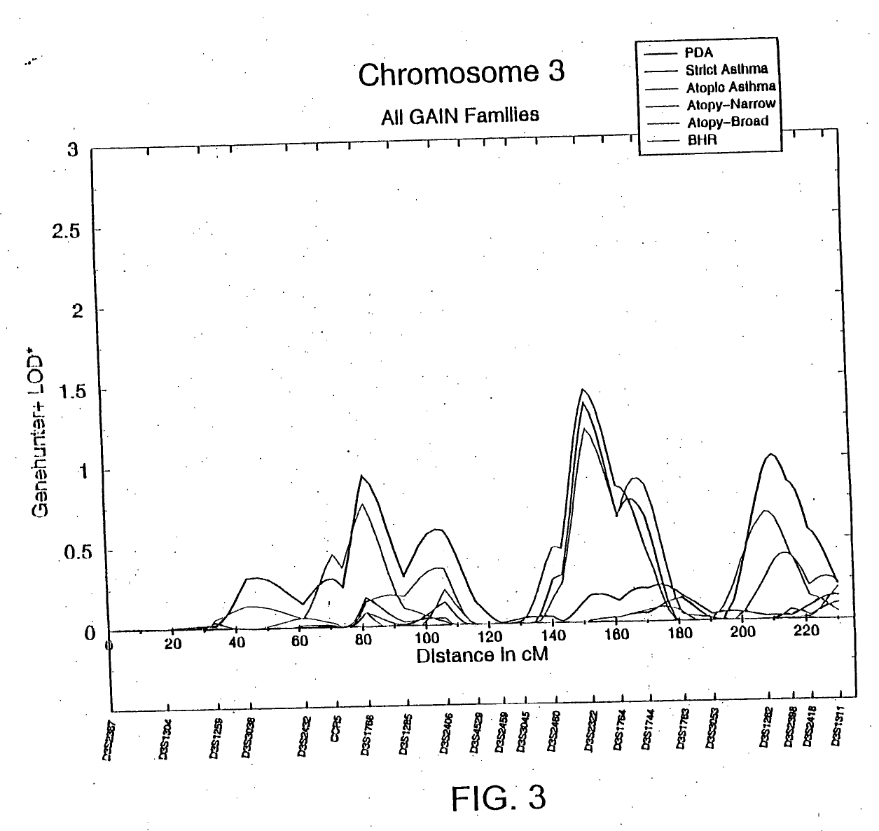

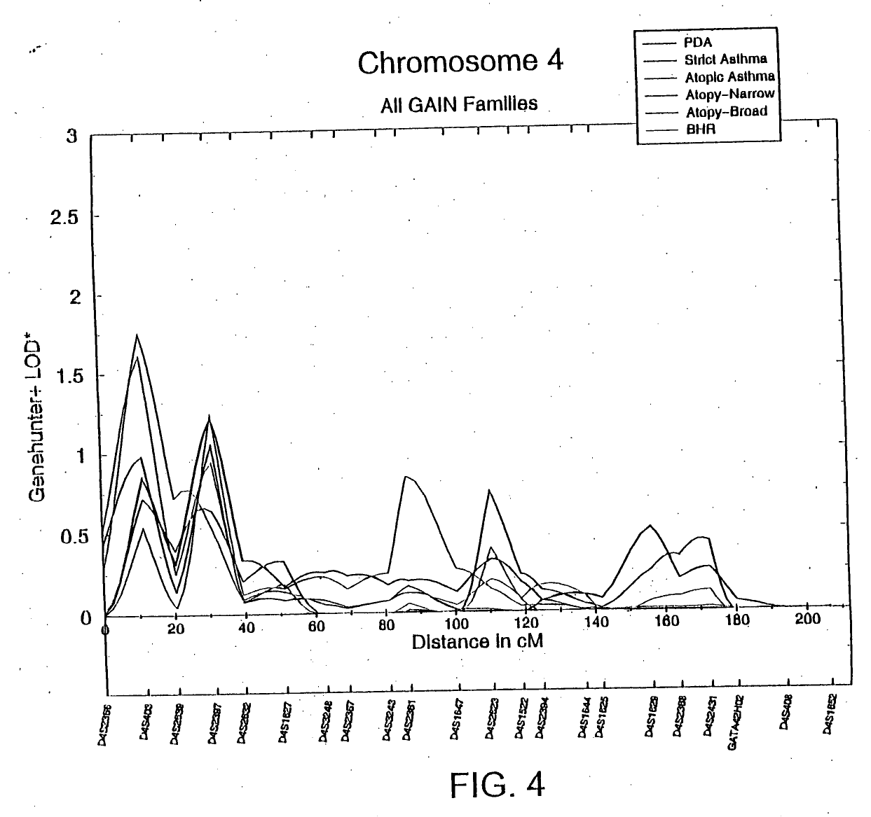

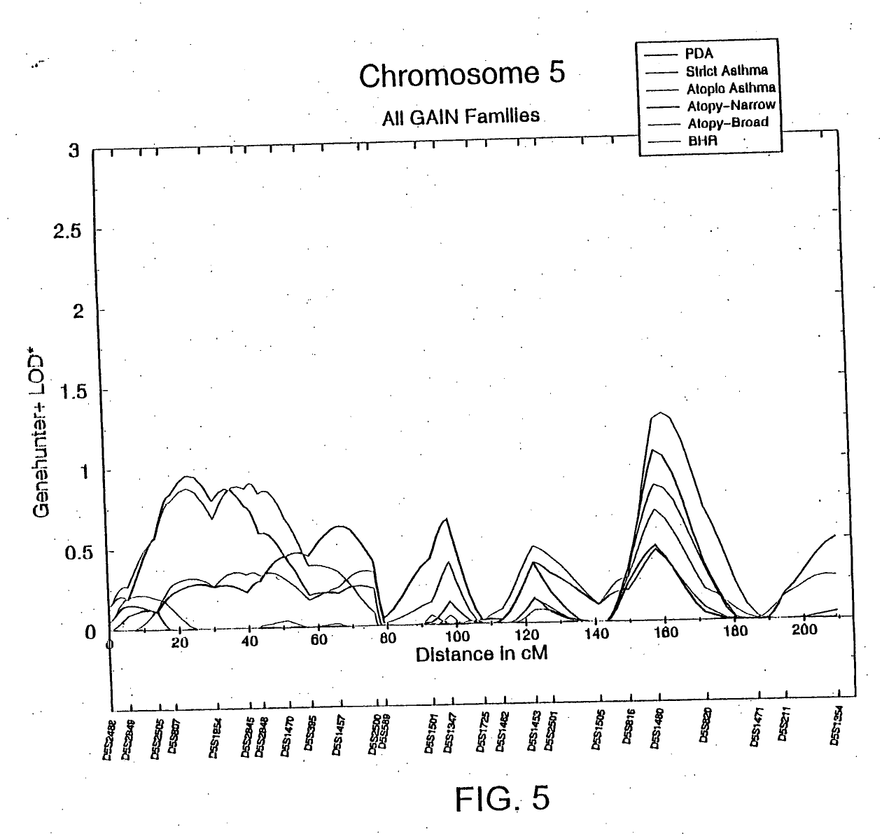

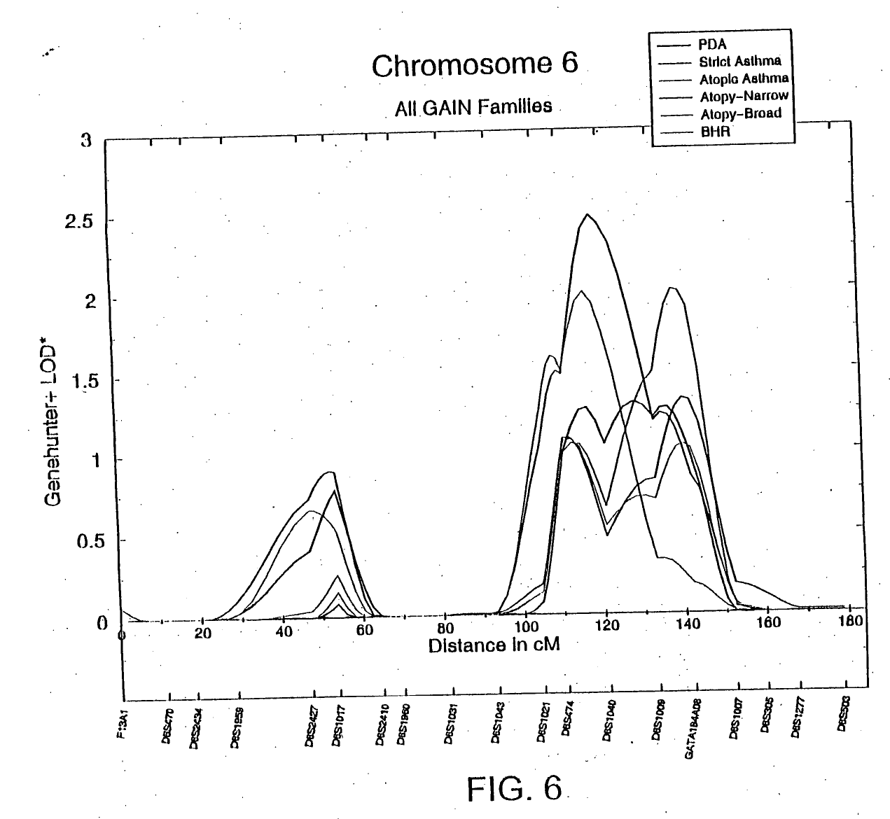

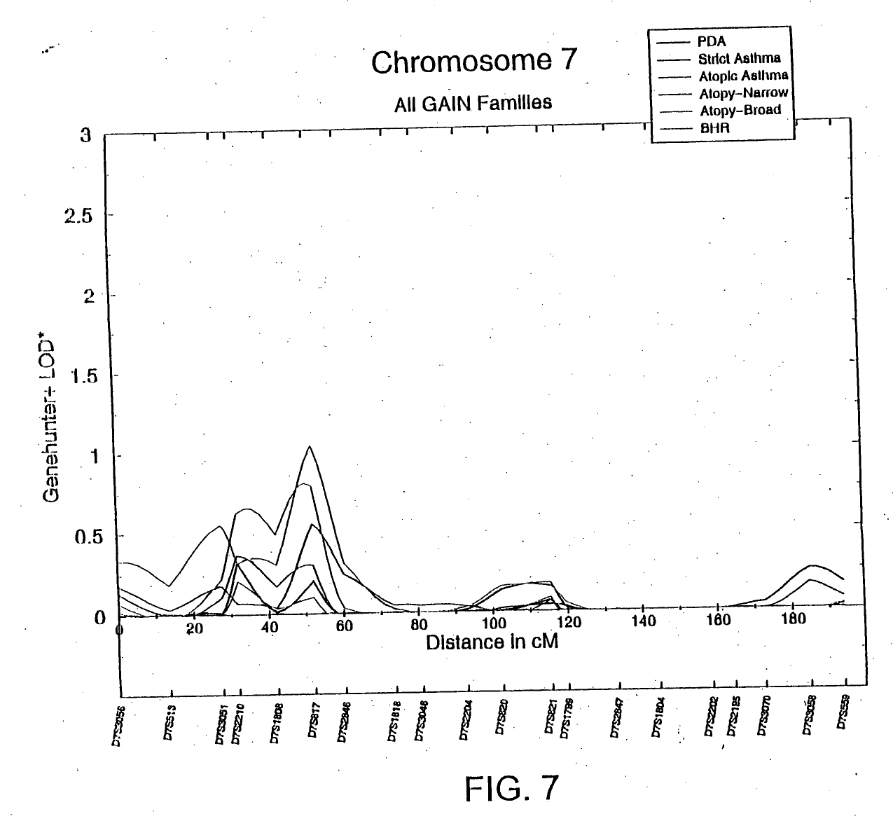

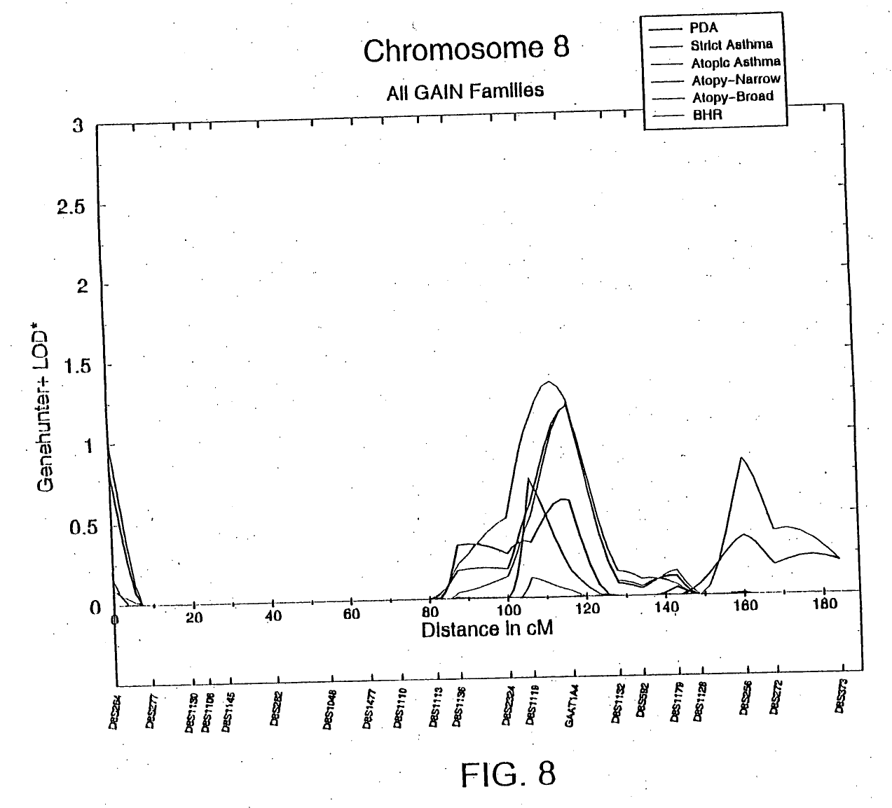

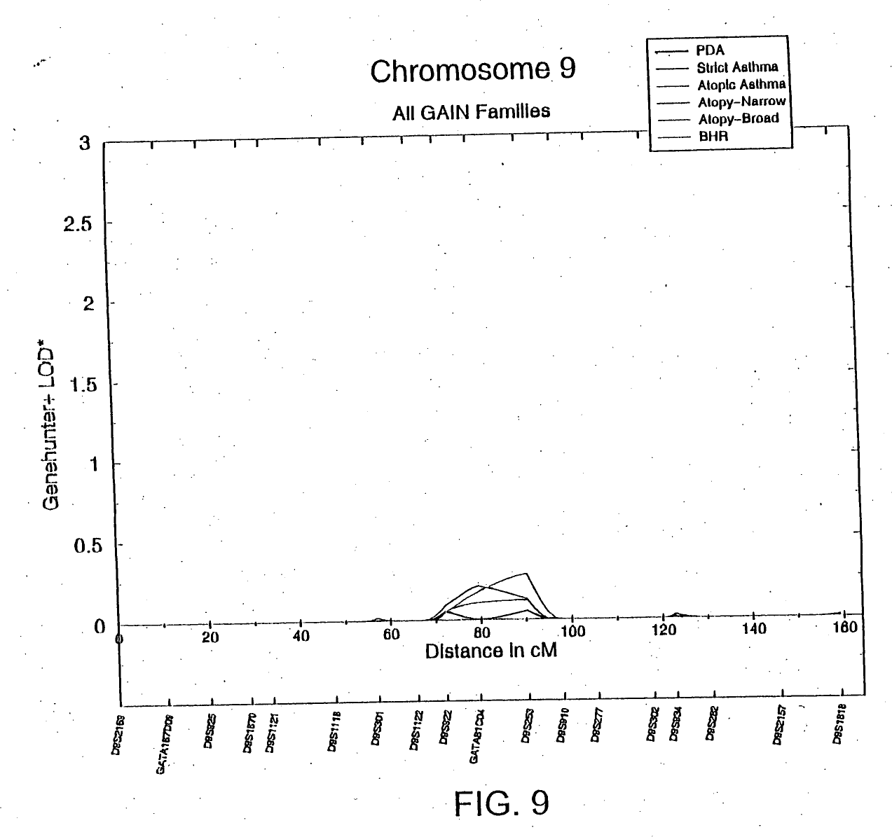

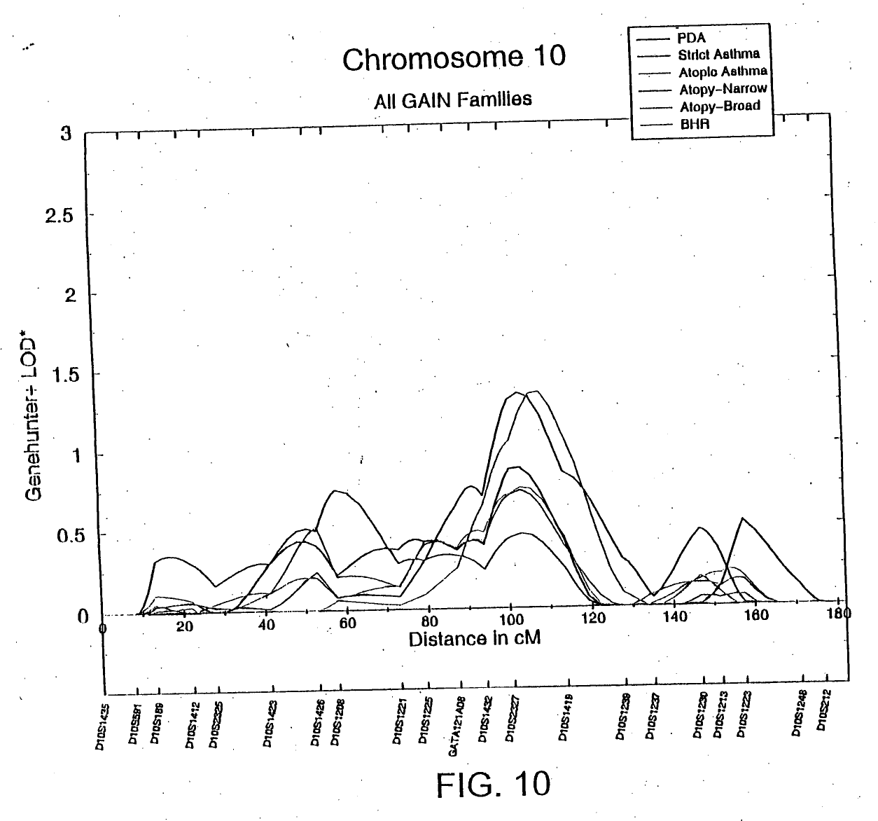

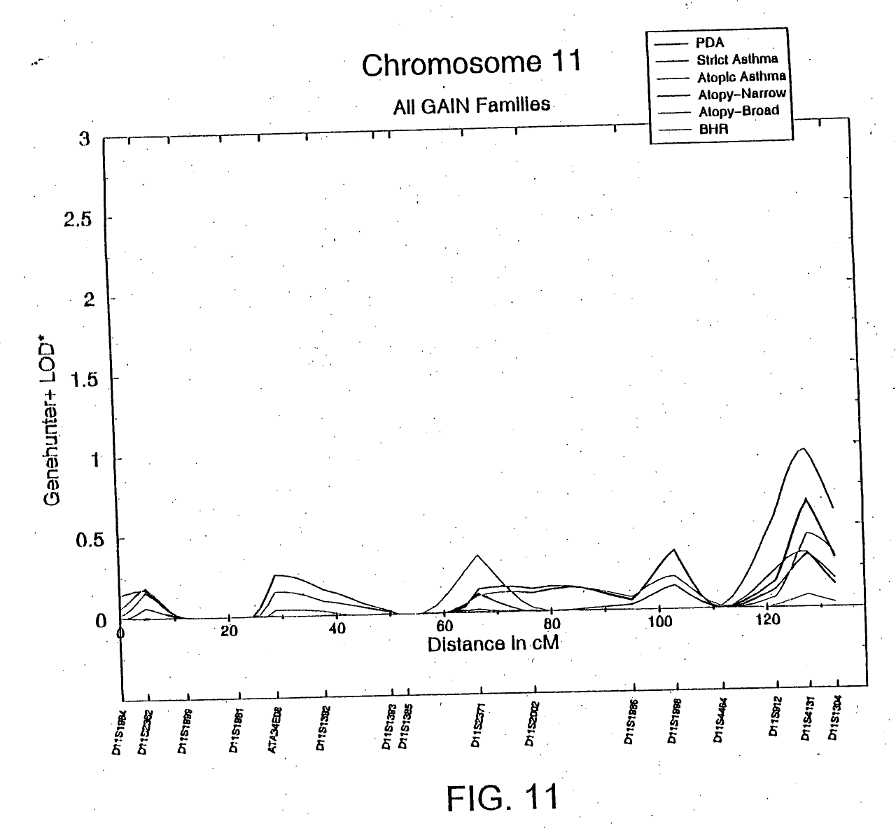

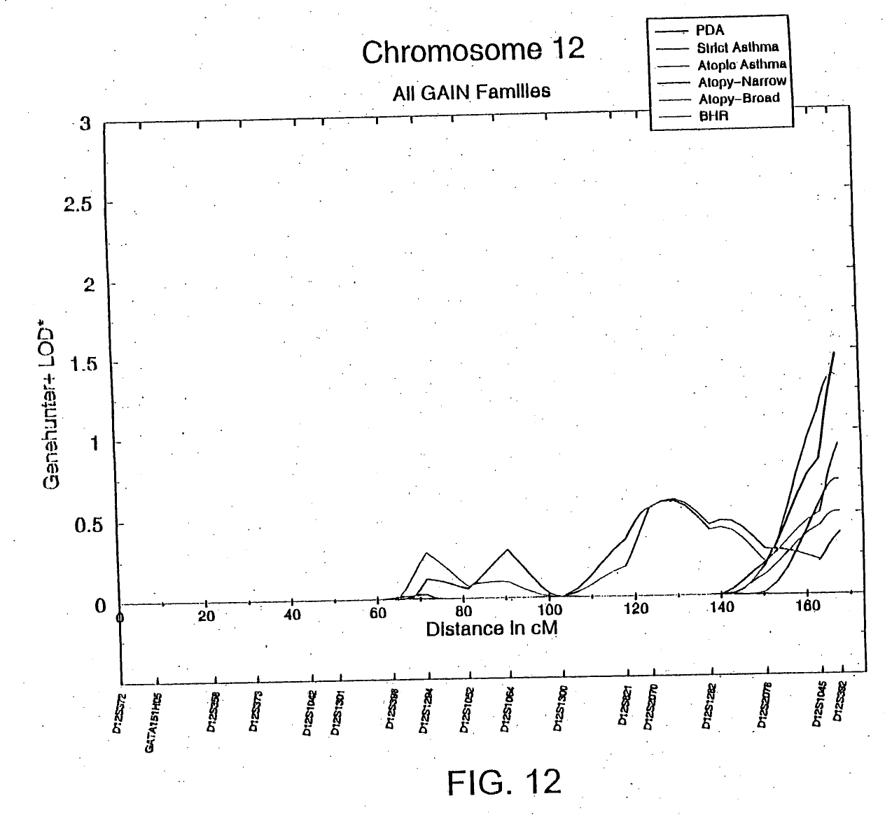

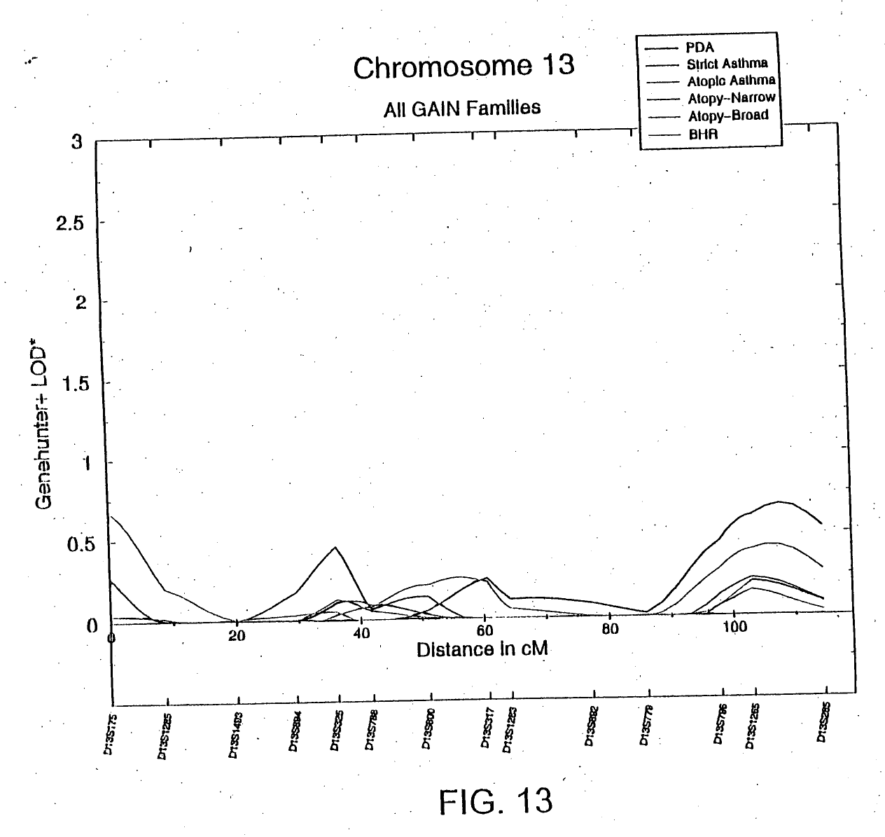

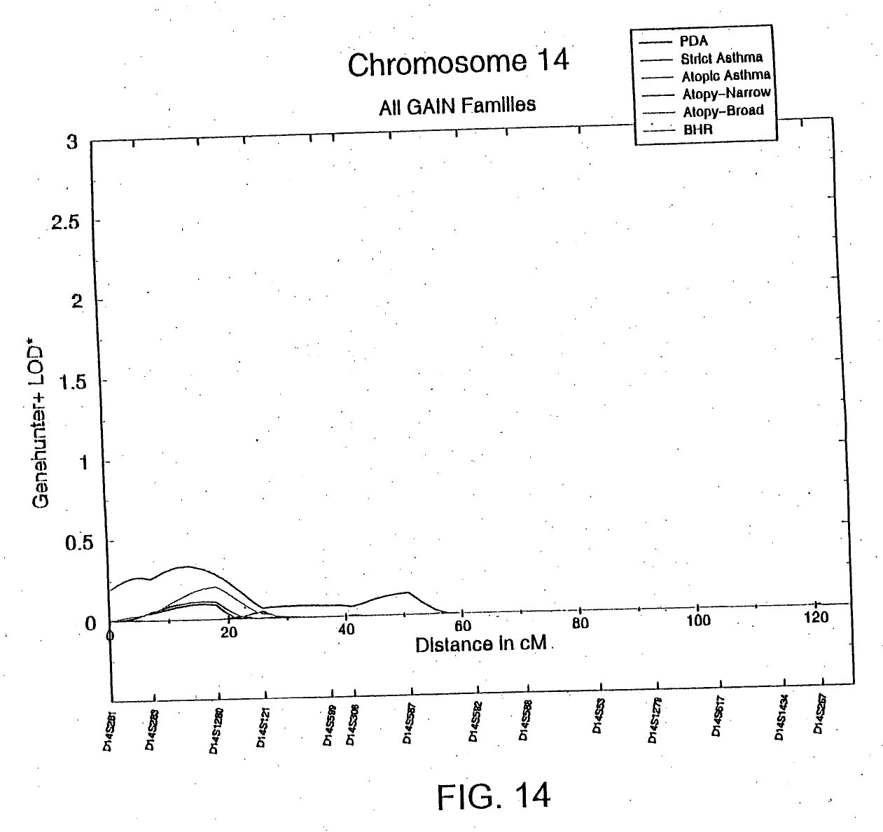

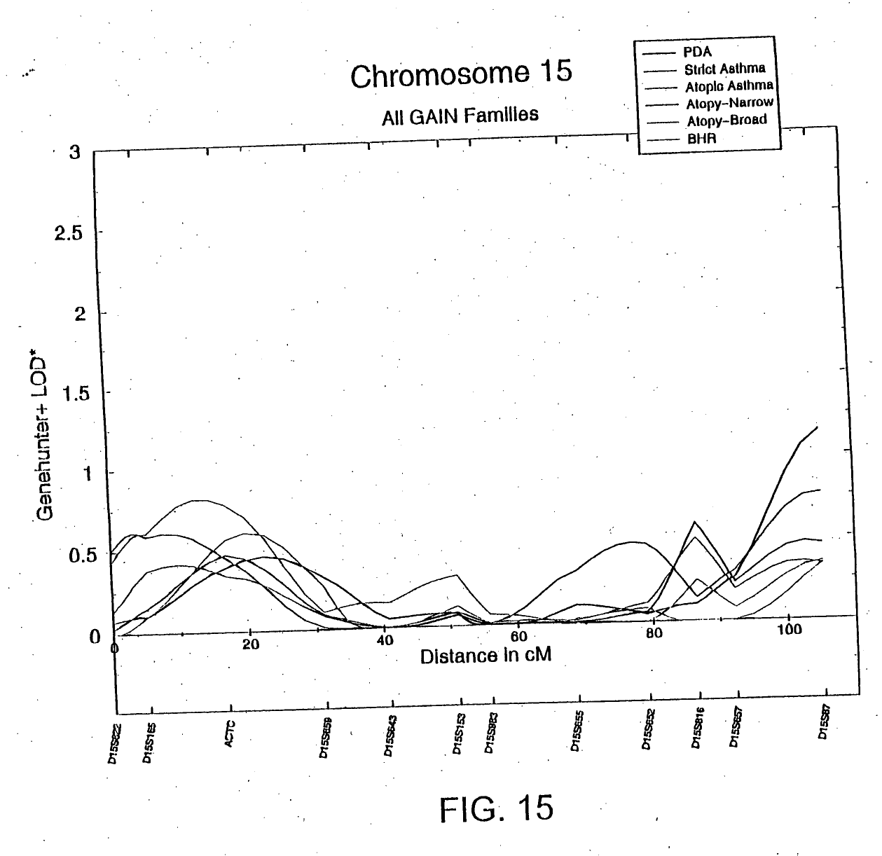

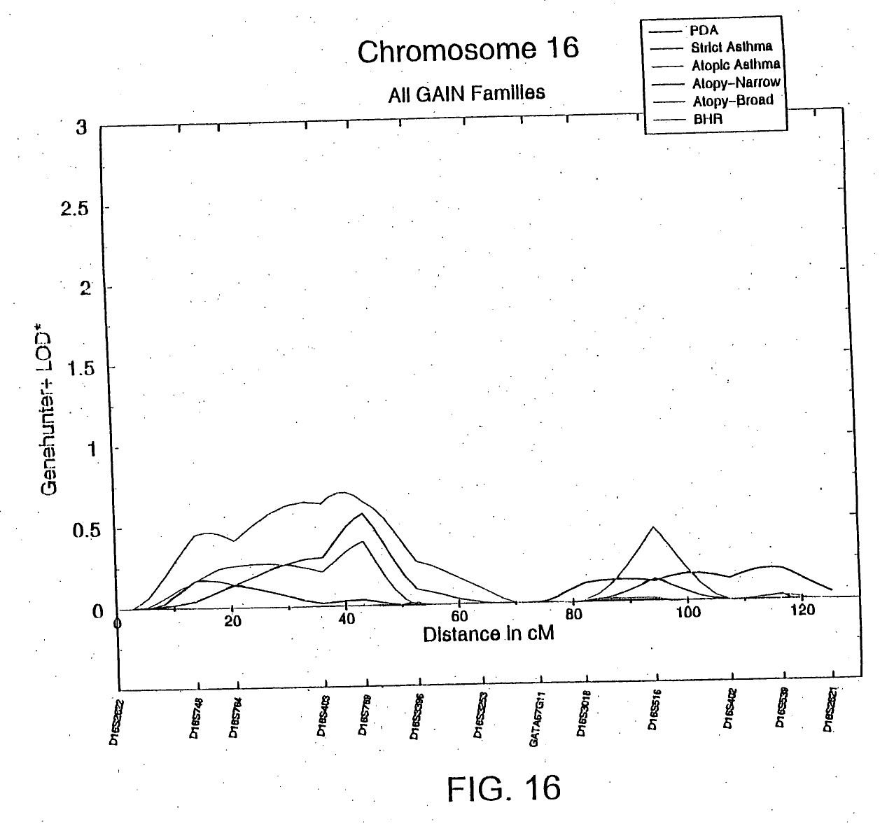

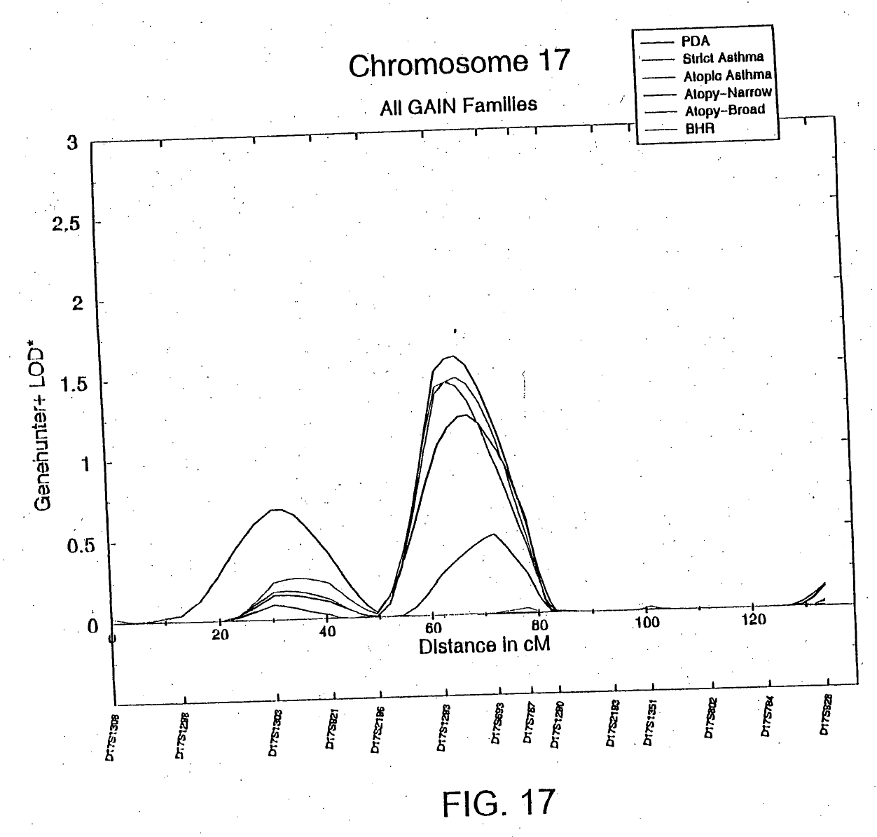

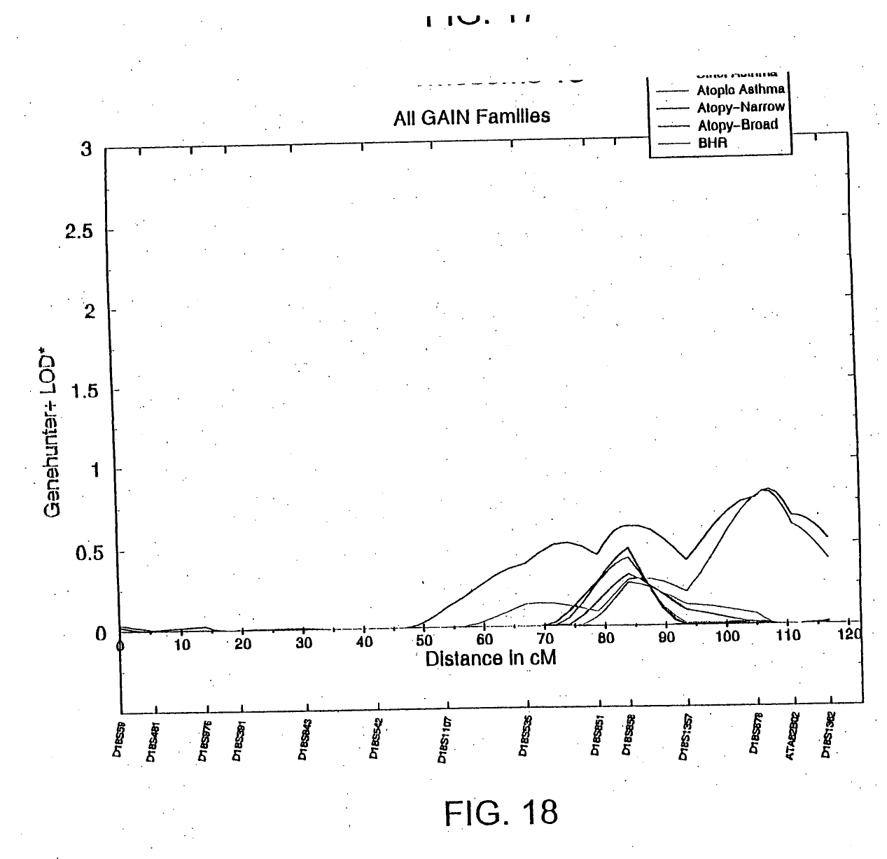

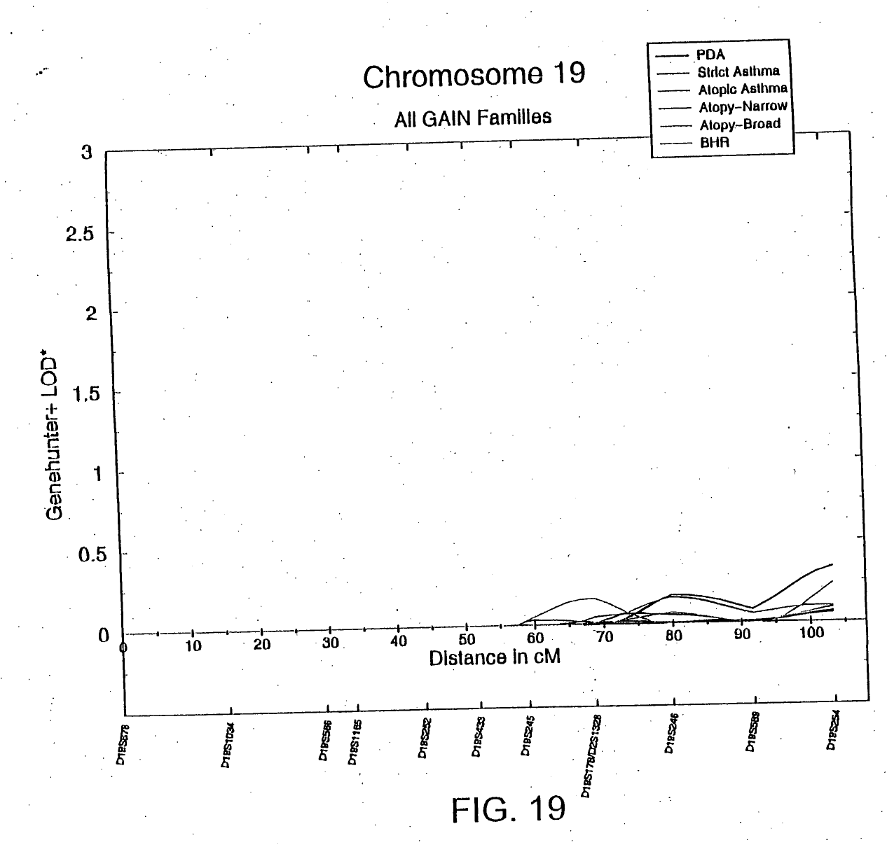

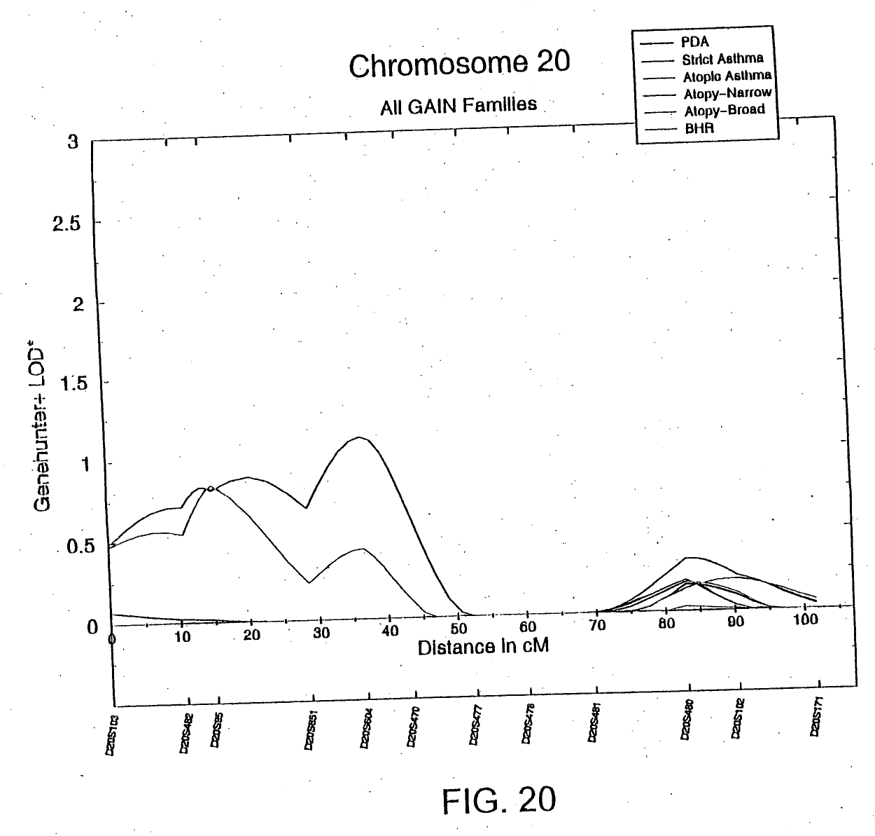

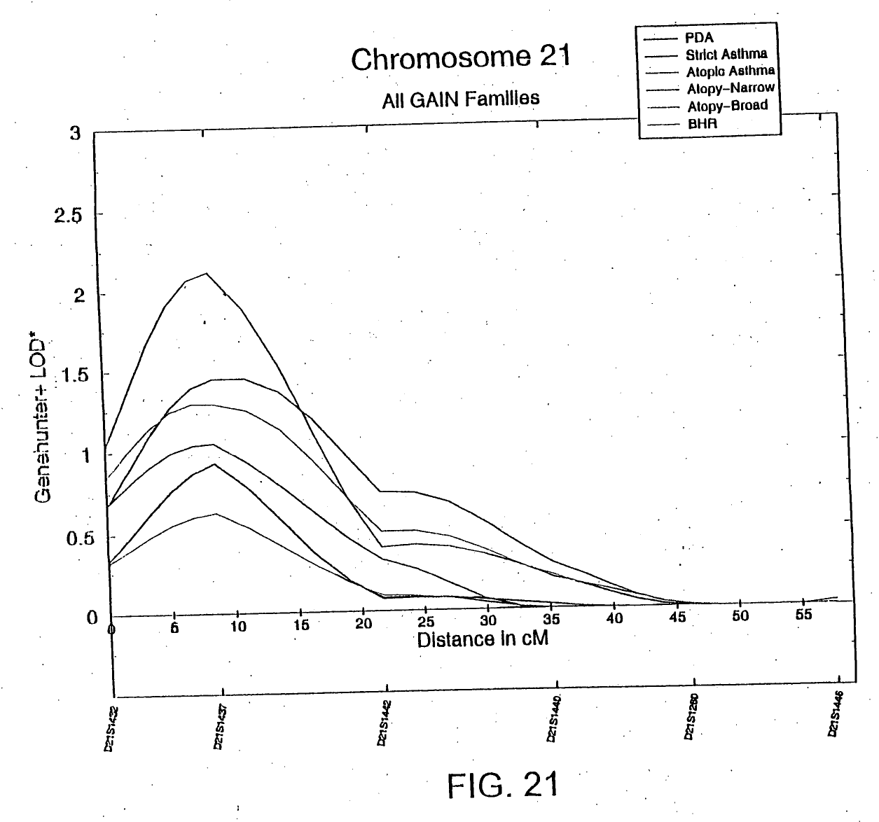

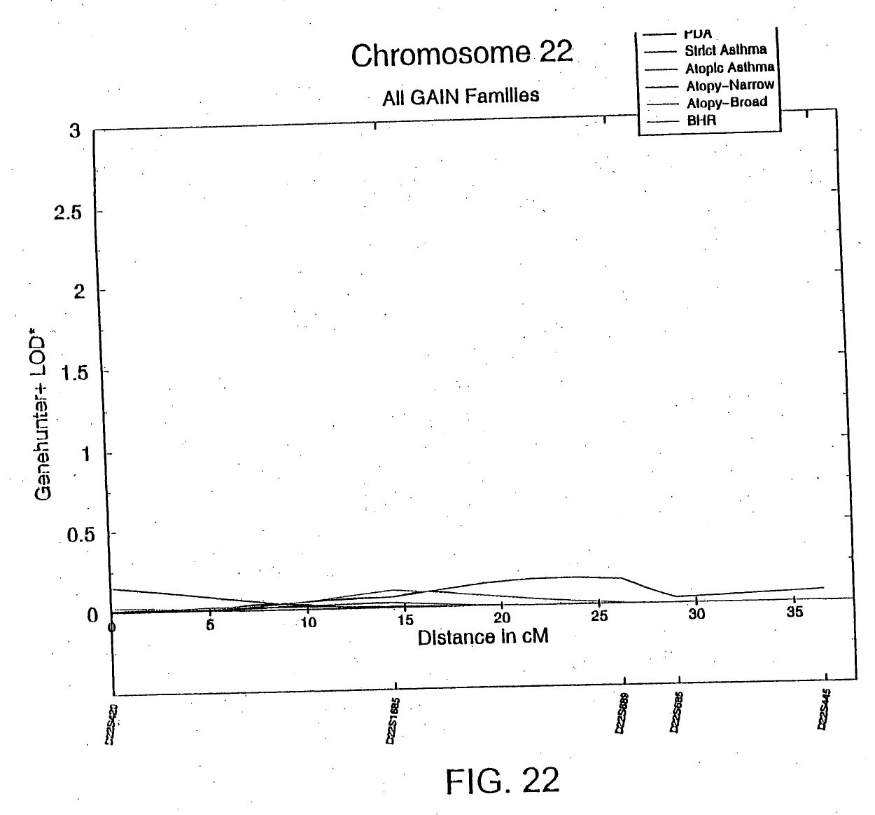

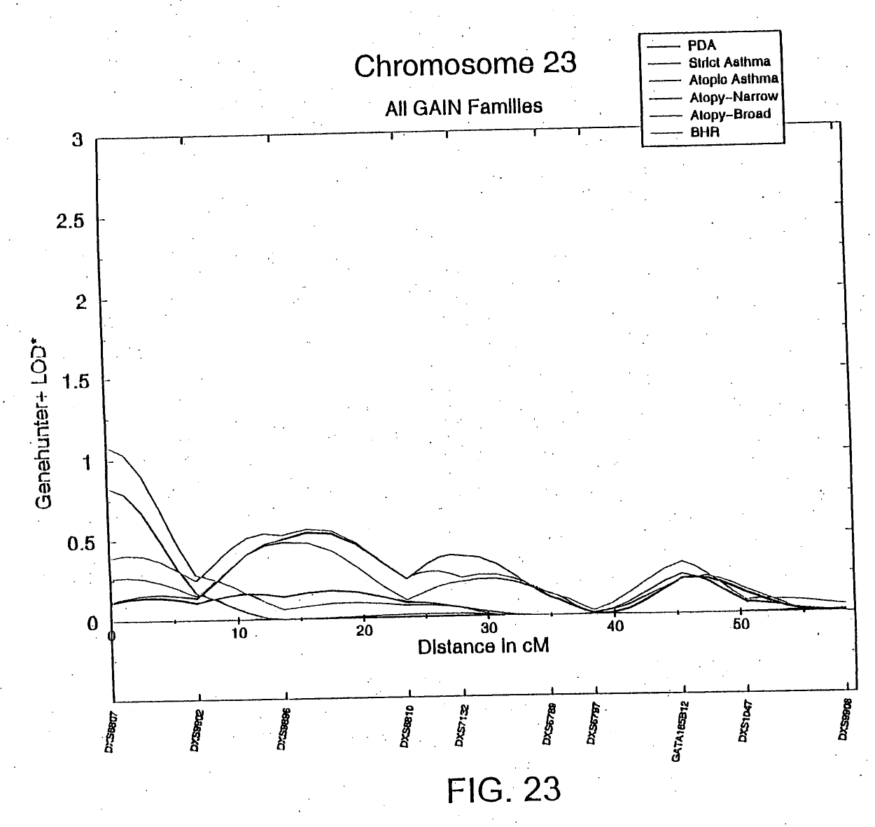

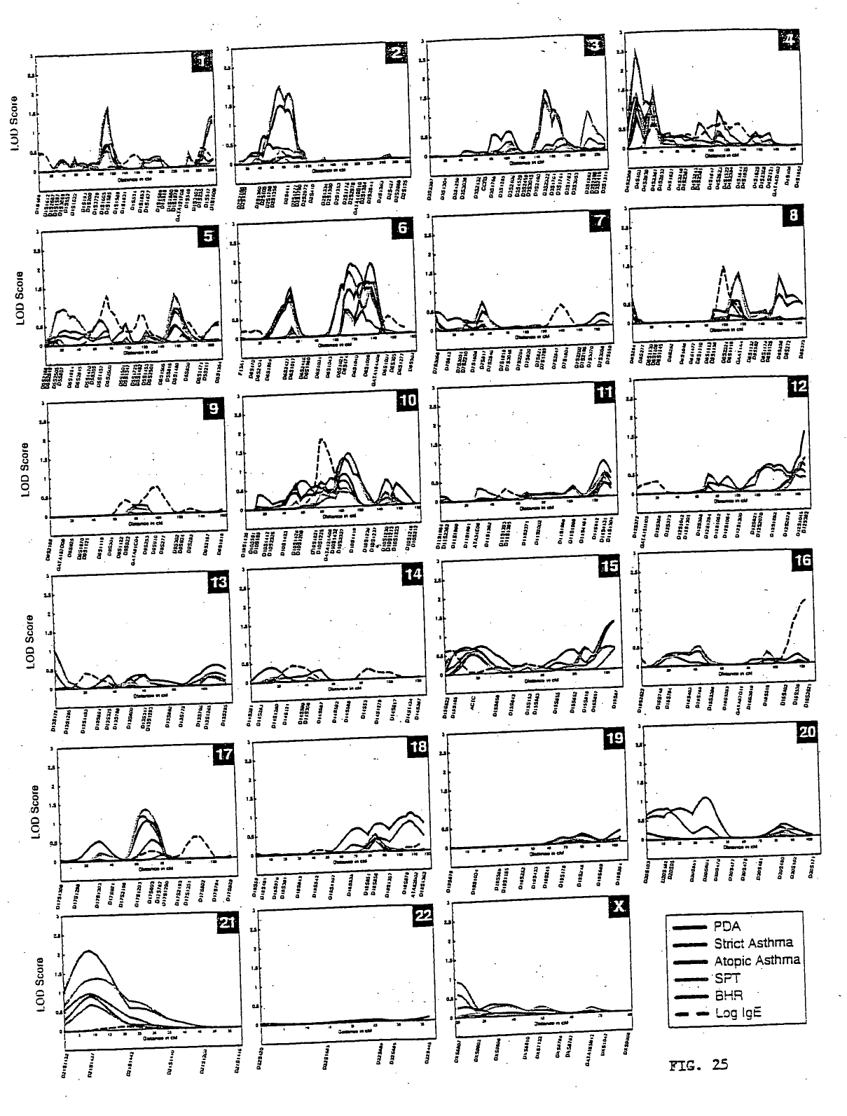

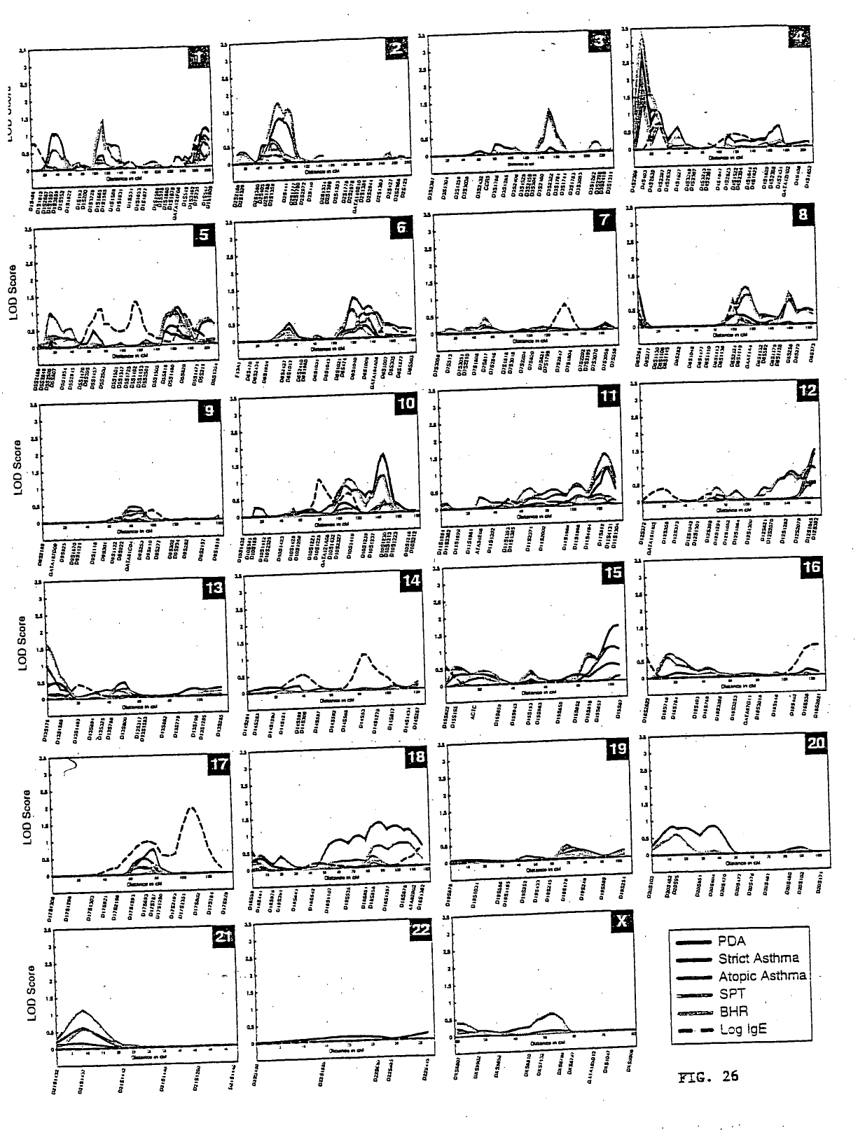

Multipoint linkage analysis using the Genehunter+ algorithm as depicted in FIGS. 1-27 were used for the discrete phenotypes. The multipoint linkage analysis confirmed all 12 regions, at least by indicating similar trends for positive lod scores. GeneHunter is a powerful software solution for optimization problems that utilizes a state-of-the-art genetic algorithm methodology. This program is used to perform multipoint linkage analysis of multiple QTLs given any pedigree and an accurate marker map. The program can be used on any quantitative data set. The program takes as input: a collection of trait/marker values, a pedigree, and a marker map. SOLAR is also able to include non-genetic factors (e.g., age, gender) as variables as well as a polygenic random effect. The program can automate the selection of background variables to be included. It begins with a general model, removing insignificant (at the 0.1 level) covariates. The result is an automated model selection process as well as a determination of the proportion of the variance attributable to background variables. (If this proportion is very high, then further analysis may not be relevant.) This automated model selection process helps to ensure a better polygenic analysis.

The output generated by SOLAR (for multipoint analysis) is the location(s) of all QTLs whose LOD score exceeds some threshold. This calculation is performed iteratively. First, the best QTL (highest LOD score) is determined. This QTL is added to the model, and the next best QTL is searched for. This process continues until no QTL remains whose LOD score is greater than some user-specified threshold. SOLAR reports the chromosome, location and LOD score for each QTL identified.

Summary of families. Two-point and multipoint analyses are found in table 1 the 365 Caucasian families analyzed through the GAIN network. These families contain 11 regions of interest (12 if counting the lod score of 1.9 on chromosome 7). Although there are some differences between the 384 family analysis and the 365 (Caucasian only) families, the results are overall very similar. A comparison of the two analyses is attached. Also noted are published chromosomal regions from other genomic screens that potentially overlap with our results; results marked in bold indicate direct overlap. Criteria for “interesting”, is at least one lod score of 2.0. Phenotypes were included as being positive in these regions if they had a lod score of at least 1.5; markers flanking the region of interest were defined as those at which the lod score was <1.0 for any phenotype. Notably, the two-point analysis of the Caucasian-only families identified regions on 6p, 7q, 12, and 15 that were not identified in the analysis of the 384 families. The multipoint analyses supported the results of the two-point analysis, and as with the two-point analysis, there were few differences between the 384 and 365 (Caucasian only) families. Specifically, a region on chromosome 4pter—D4S2632 increases to a multipoint lod score of 2.4 with the BHR phenotype (two-point lod scores were hovering around 1.4) and this peak is supported by a multipoint lod score in the same region of approximately 2.0 for the atopy-broad phenotype.

TABLE 1

| Chromosome | Marker | Region | bp × 10⁶ | cM^{a} |
|---|---|---|---|---|
| 1 | D1S200 | 1p32.1 | 58.728 | 82.41 |
| | D1S1631 | 1p13.2 | (112) | 136.88 |
| 5 | D5S2845 | 5p13.3 | 30.481 | 36.25 |
| | D5S589 | 5p11 | (45) | 73.35 |
| 6 | D6S502 | 6q16.1 | 99.025 | 101.55 |
| | D6S503 | 6q27 | 175.160 | 184.51 |
| 7 | D7S2195 | 7q35 | 148.121 | (165) |
| | D7S559 | 7q36.3 | 161.084-.085 | (185) |
| | D7S1808 | 7p15.1 | 28.870 | 41.69 |
| | D7S2846 | 7p14.1 | 38.965 | 57.79 |
| 8 | D8S1113 | 8q12.1 | 58.671 | 77.89 |
| | D8S1132 | 8q23.1 | 106.936 | (129) |
| 9 | D9S922 | | | |
| | D9S253 | | | |
| 10 | D10S1221 | 10q11.23 | 57.933 | (73) |
| | D10S1132 | 10q22.1 | 77.832 | 93.92 |
| 15 | D15S822 | 15q11.2 | 24.177-.272 | 12.30 |
| | D15S643 | 15q21.3 | 58.205 | 52.33 |
| 17 | D17S1298 | 17p13.3 | 3.782-.837 | 10.72 |
| | D17S787 | 17q22 | 57.024 | 74.99 |
| 19 | D19S1034 | 19p13.3 | 8.287 | 20.75 |
| | D19S1165 | 19p13.2 | 15.287 | 36.22 |
| 21 | | pter | 0 | 0 |
| | D21S1442 | 21q21.3 | 25.397 | 24.73 |

^{a}Marshfield sex-averaged genetic map location

Multipoint analysis was able to determine the following. First, the regions on chromosome 17 (region based on multipoint is D17S2196-D17S1290) and 21 (region based on multipoint is pter—D21 S1442) were able to be narrowed. In general, the multipoint lod scores were lower than the highest two-point lod score reported in the region of interest. In the multipoint analysis, the same region was implicated when compared with the two-point analysis. It is noted however that the maximum lod score from the strict asthma phenotype increases from approximately 1.9 in the two-point analysis to almost 2.5 in the multipoint analysis for this region on chromosome 6. Based on the multipoint analysis, this region on chromosome 6 spanned by D6S1043-D6S1007 appears to be most promising with the highest overall multipoint lod score.

Of additional interest is that the region on chromosome 5 spanned by D5S2848-D5S589 identified with a maximum lod of 2.0 in two-point analysis now has multipoint lod scores <1.0 for all phenotypes. A region more distal on chromosome 5 yields a multipoint lod score for the atopy-broad phenotype of approximately 1.28. This region demonstrates direct overlap with the 5q31-q33 region spanning the cytokine cluster implicated previously in other asthma/atopy studies. An overall view of all sites in overlap is shown below, with overlap sites in bold.

TABLE 2

| Chromosome | Marker | Region | bp × 10⁶ | cM^{a} |
|---|---|---|---|---|
| 1 | D1S468 | 1p36.32 | 3.547 | 4.22 |
| | D1S597 | 1p36.21 | 12.249-520 | 29.93 |
| | D1S3669 | 1p36.13 | 17.076 | 40.32 |
| | D1S239 | 1p13.2 | 112.020 | 139.02 |
| | D1S228 | 1p36.21 | 12.318 | 29.93 |
| | D1S207 | 1p22 | (90) | 113.69 |
| | D1S221 | 1p13.1 | 115.494 | 142.24 |
| | D1S502 | 1p12 | 118.643 | 146.53 |
| | D1S419 | 1q41 | 225.293 | 232.81 |
| | D1S229 | 1q42.12 | 227.946 | 237.73 |
| 5 | D5S1480 | 5q33.1 | 153.373 | 147.49 |
| | D5S1462 | 5q14.3 | 93.543-94.040 | 105.29 |
| | D5S1453 | (5q22.1) | (111) | (115) |
| | D5S2014 | 5q33.2 | 156.580 | 153.17 |
| | D5S2111 | 5q35.3 | 181.482 | 187.81 |
| | | 5q31-33 | | |
| 6 | MHC | 6p22.1-21.1 | | |
| | TNF | 6p22.3-21.31 | | |
| | | 6p25 | | |
| | | 6p21 | | |
| 7 | D7S517 | 7p22.2 | 4.280 | 7.44 |
| | D7S531 | 7p22.3 | 3.004 | 5.28 |
| | D7S488 | 7p15.3 | 20.256 | 29.28 |
| | D7S528 | 7p14.1 | 38.529 | 57.79 |
| | | 7p14-15 | | |
| 8 | D8S1110 | 8q11.22 | (51) | 67.27 |
| | D8S1477 | 8p11.23 | 32.671 | 60.34 |
| | D8S1136 | 8q12.3 | (64) | 82.26 |
| | D8S2324 | 8q13.3 | 74.065 | 94.08 |
| | D8S529 | 8q24.22 | 135.034 | 148.12 |
| | | 8q23-21 | | |
| 9 | D9S925 | 9p21.3 | 19.601 | 32.24 |
| | D9S922 | 9q21.31 | 72.498 | 80.31 |
| | D9S938 | 9q31.1 | 96.701 | 135.73 |
| | D9S156 | 9p22.2 | 17.473 | 30.61 |
| | D9S283 | 9q22.1 | (81) | 94.85 |
| | D9S1784 | 9q31.2 | 98.775 | 111.99 |
| | | 9p22 | | |
| | | 9q13 | | |
| | | 9q23 | | |
| | | 9q32 | | |
| 10 | D10S581 | 10q21.3 | 68.106 | 82.5 |
| | D10S537 | 10q22.1 | 75.264 | 91.13 |
| 15 | D15S1042 | 15q13.2 | 31.742 | 32.58 |
| | D15S127 | 15q26.1 | 88.344 | 86.81 |
| | D15S120 | 15q26.3 | 95.952 | 112.58 |
| | | 15q22 | | |
| 19 | D19S534 | 19p13.3 | 10.083 | (26) |
| | D19S922 | 19p13.3 | 9.050-10.894 | 26.37 |
| | D19S884 | 19p13.3 | 9.062-10.881 | 26.37 |
| | D19S216 | 19p13.3 | 6.993 | 20.01 |
| | D19S567 | 19p13.3 | (9) | 25.17 |
| | D19S120 | 19p13.3 | 4.230 | 10.97 |
| | D19S536 | 19p13.3 | ^{c} | ^{c} |
| | D19S413 | 19p13.2 | (12) | 32.39 |
| | D19S900 | 19q13.32 | 60.595 | 67.37 |
| | D19S540 | 19q13.32 | (63) | 70.14 |
| | D19S178 | 19q13.32 | 60.841 | 68.08 |
| | | 19p13 | | |
| | | 19q13 | | |
| 21 | D21S1262 | 21q22.3 | 42.640 | (54) |
| | D21S1440 | 21q22.13 | 35.178 | 36.77 |
| | | 21q21 | | |

Markers and Regions in bold denote potential overlaps with regions proposed to be asthma and/or atopy markers.
^{a}Marshfield sex-averaged genetic map location.
^{b}STS map location for D5S1470 is not in region proposed. However, the Marshfield map location for this marker indicates that this marker is in the region proposed.
^{c}STS marker name is obsolete.

Using a lod score of 2.0, multipoint analysis did not identify any new regions not previously identified in the two-point analysis. However, if the criterion is relaxed to a multipoint lod score of at least 1.5, then a region on chromosome 4 (pter—D4S2632) is implicated, with the BHR and atopy-broad phenotypes yielding multipoint lod scores >1.5.

Other interesting regions include those spanned by D8S1136-D8S1132 (max lod of 2.5 with positive results for 5 phenotypes); 21pter-21qter (max lod score of 2.5 with positive results for three phenotypes), and D15S816-D15S87 (max lod score of 2.2 with positive lod scores for 3 phenotypes). The highest lod score of 2.8 was obtained for IgE, spanned by D10S1221-D10S1432.

Overall, between the two sets of analyses there are 16 regions of interest identified (+2 if the regions on chromosome 7 with lods scores slightly less than 2.0 are included) from either two-point or multipoint analysis.

Multipoint linkage analysis of quantitative traits IgE and slope of the BHR response (SOLAR). The multipoint lod scores for the quantitative traits IgE and slope of BHR response are consistent between the 365 (Caucasian-only) and 384 (full dataset) families. The regions identified via two-point analysis on chromosomes 8, 10, and 16 for IgE continue to remain positive; however, the overall scores decrease. The highest lod scores for IgE remains the region spanned by D10S1221-D10S2327 with a peak multipoint lod score of approximately 1.7 in both series. The slope of BHR response continues to be a relatively uninformative phenotype in these series.

Multipoint linkage analysis of discrete traits using SOLAR. In addition to GENEHUNTER, SOLAR was used for multipoint analysis of discrete traits. The results were generally consistent between the analytic approaches, with a few notable exceptions. The most notable exception is that SOLAR identified a region on chromosome 9 that was not identified via GENEHUNTER. In the 384 families, the peak multipoint lod score (SOLAR) for the atopy-narrow phenotype was approximately 2.0 at D9S253 (slightly lower in the 365 Caucasian families at 1.89; the interval is large and spanned by D9S1118 and D9S277.

The regions on chromosomes 5 and 17 demonstrated stronger (higher lod scores) and/or broader peaks with SOLAR than those identified via GENEHUNTER. The most substantial of these differences are on chromosomes 5p and 17. For chromosome 17, the peak GENEHUNTER multipoint lod was approximately 1.59 in the region spanned by D17S2196 and D17S2190 in the 384 families (Caucasian+non-Caucasian) for the phenotypes including atopy (atopic asthma, atopy-broad, atopy-narrow); using SOLAR, this region was most interesting for the atopic asthma and atopy-narrow phenotypes and yielded a multipoint lod score of approximately 2.2 in a much broader region. Using SOLAR in the 365 Caucasian families, the atopy-narrow phenotype generated a lod score of approximately 2.5 in this broader region. For 5p, in the 384 families GENEHUNTER identified a peak multipoint lod score of approximately 1.0 with the atopy broad and narrow phenotypes but SOLAR identified a lod score of approximately 2.0 (atopy-broad phenotype) and 1.67 (atopy-narrow) in the region bounded by D5S2845 and D5S1725. Slightly lower multipoint lod scores in this interval are observed for these phenotypes in the 365 Caucasian families using SOLAR.

SOLAR multipoints did confirm the region on chromosome 2 identified via GENEHUNTER for the BHR phenotype, but did not confirm the regions on chromosomes 1, 4, and 6.

Additionally, individualized homogenous populations have led to the additional discoveries of asthma and atopic markers in that specific population.

Greek Families

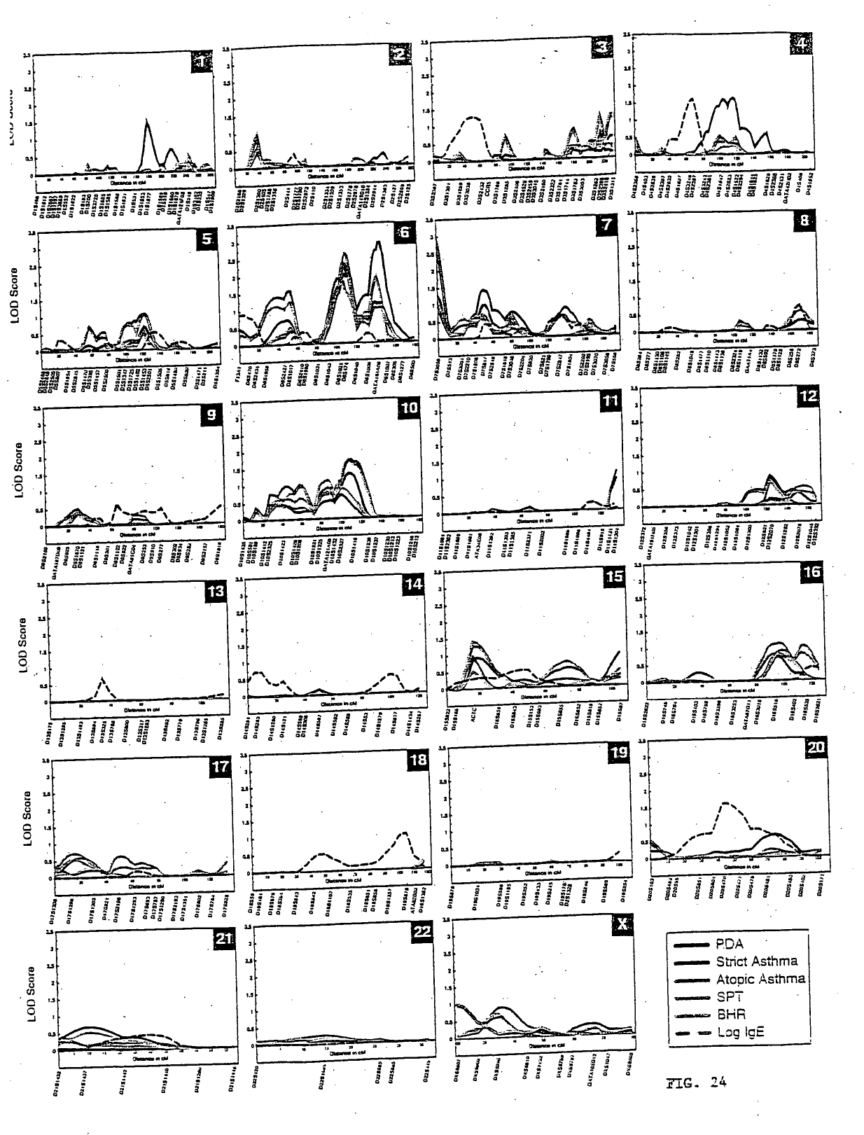

A multipoint lod score of 3.0 in the Greek population for the phenotype of Physician Diagnosis of Asthma (PDA) was identified on 6q spanned by D6S1043 and D6S1007. This region on 6q representd a novel susceptibility locus in the Greek population. FIG. 24 illustrates that 107 genes and ESTs have been identified in the region. Clinically, the Greek families have statistically significant differences in median IgE levels with the Greek population being lower than others, and a higher proportion of families with at least one asthmatic parent.

Norwegian Families

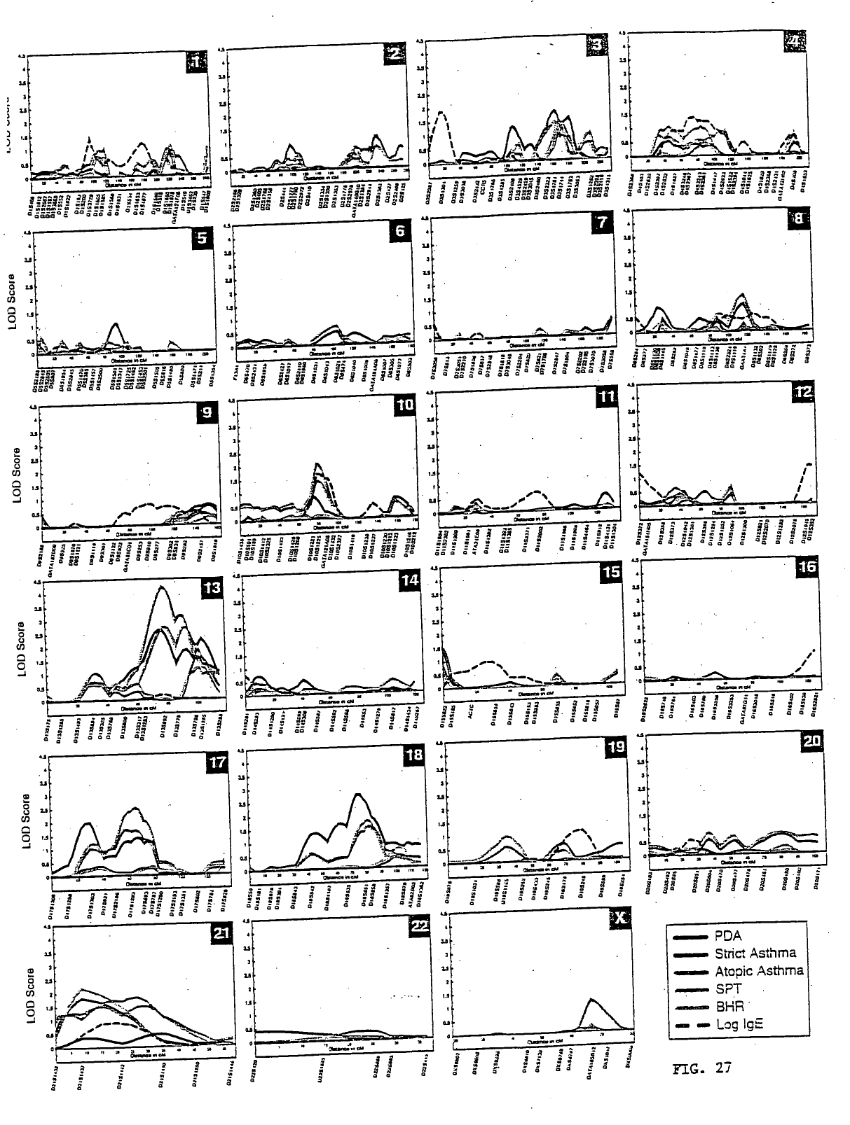

A multipoint lod score of 4.2 was identified in the Norwegian series in a region on chromosome 13 spanned by D13S1283 and D13S779 with the phenotype atopic asthma. The phenotypes of physician diagnosis of asthma (PDA) and narrow atopy (at least one positive skin prick test) also support this interval with maximum multipoint lod scores of 2.5 and 2.4, respectively. In this region, 176 genes and ESTs have been identified as shown in FIG. 27. This region overlaps with three other reports.

Chromosome 11 (ASTH_I/J Region)

A total of 37 candidate gene markers of individuals in 365 Caucasian-only pedigrees were analyzed for twopoint linkage and association. Modified multipoint linkage analysis was performed to allow the joint consideration of all SNPs. Additionally, the 365 families were analyzed separately in 8 by-center groups. Analyses were done using 6 discrete traits: Physician diagnosis of asthma (PDA), Bronchial hyperresponsiveness (BHR), Strict asthma, Atopic asthma, Atopy-narrow (SPT≧3 mm), Atopy-broad (SPT≧3 mm or IgE>100 IU) and 2 quantitative traits: log of IgE and slope of the methacholine dose-response curve.

SNP data was investigated for errors utilizing the CHROMIPC option of CRIMAP to identify families that may harbor numerous recombination events within this small region. One family (ABD 2) showed one individual with 5 recombination events within the chr11 gene and so it was excluded from all analyses. Families with 1 (n=12) or two (n=9) recombination events were retained in the analysis.

All twopoint lod scores were obtained using three methods: A Hetlod score which was the maximum obtained assuming heterogeneity among families of the dominant and recessive parametric models for a given marker corrected for multiple testing, a Lod* from the ASM program run after Genehunter+, and a Lod score from the SOLAR program. The quantitative traits were run with the SOLAR software alone.

Three regions in the overall data set produced lod scores ≧1.5. All of these loci were located in the ASTH_I/J region, with none found on the EDN1 or CYSLT2REX1 areas. The maximum lod score obtained was 1.58 for asth.sequana93 with the trait Atopy-narrow. Two additional traits (Atopy-broad and log IgE) were over 1.0 for this marker. The second region was asth.sequana17 with a lod score of 1.54 for Atopy-broad, which also had a lod score of over 1.0 for Atopy-narrow. Finally, asth.sequana45 had a maximum lod score of 1.53 for Atopy-broad, with three additional phenotypes having lod scores >1.0 (Atopy-narrow, Atopic Asthma, and BHR).

Twopoint lod scores were also generated by site. Three sites (DUK, LEI, and SHF) generated no lod scores over 1.0 individually. The largest twopoint lod score by site was found using ABD and the trait BHR with a lod score of 3.76 for pol3365, with a lod score of 2.38 at pol3367. OSL had one lod score of approximately 2.0 (1.95) for log IgE at pol3364. PER had 3 lod scores of approximately 1.0 for IgE. STO had a maximum lod score of 1.52 for Atopic Asthma at pol3205. The highest lod score generated by THS was for PDA, 1.67 at pol3204.

Modified multipoint lod scores to determine maximum lod score for the asthmaI/J region using Genehunter+/ASM and Solar were generated for the ASTH_I/J region of chromosome 11. Multipoints were calculated for the total data set and for each site with a lod score ≧1.0 for the region. ABD, OSL, STO, and THS were analyzed with Genehunter+/ASM for the discrete traits, and ABD, OSL, PER, STO, and THS for log IgE. Three regions generated lod scores ≧1.5, all of which were site-specific. The highest lod score obtained across all sites and phenotypes was for Oslo and log IgE, approximately 2.3. Analysis of BHR with ABD found a lod score of approximately 1.8. Finally, PDA with THS obtained a lod score of 1.5.

Transmit analyses using PDT were run for each of the 6 discrete traits to look for association for the entire data set and by site. Analyses were run both as twopoints and as marker subsets. No trait had significant results when looking at the total data set. Scattered significant results were seen when looking at p-values by site, with a majority of these found for DUK, LEI, and SHF, none of which were significant individually for twopoint lod scores. These are also the 3 smallest subsets, with approximately 30 families each and we attribute these to small sample size; however, it is important to note that more data are available from each of these sites that could be used to augment the analysis. This data suggests that the asthmaI/J regions may be involved in the genetic basis of asthma. The GAIN study illustrated a marker may be within a segment of chromosome 11 bordered by D11S4131-qter or within a segment of chromosome 11 bordered by D11S912-qter. The marker for chromosome 11 bordered by D11S912-qter is primarily for Caucasians.

In the specification, there has been disclosed typical preferred embodiments of the invention and, although specific terms are employed, they are used in a generic and descriptive sense only and not for purposes of limitation of the scope of the invention being set forth in the following claims.

1. A method of screening a subject for asthma and/or atopy comprising:
detecting the presence or absence of at least one or more markers linked to asthma and/or atopy, wherein the presence of said marker indicates that the subject is afflicted with or at risk of developing asthma and/or atopy, and wherein said marker is selected from the group consisting of D1S200, D1S1631, D5S2845, D5S2848, D5S589, D6S502, D6S503, D6S1043, D6S1007, D7S2195, D7S559, D7S1808, D7S2846, D8S1113, D8S1132, D9S922, D9S253, D10S1221, D10S1432, D11S912, D11S4131, D13S1283, D13S779, D15S822, D15S643, D17S1298, D17S787, D19S1034, D19S1165 and D21S1442 and markers within three centimorgans thereof.

2. The method according to claim 1, wherein said method is a diagnostic method.

3. The method according to claim 1, wherein said method is a prognostic method.

4. The method according to claim 1, wherein said subject is human.

5. A method for diagnosing a subject as being asthmatic, or as having a predisposition to asthma comprising:
determining the presence or absence of an allele of a polymorphic marker in the DNA of the patient, wherein (i) the allele is associated with a phenotype of asthma, and wherein (ii) the polymorphic marker is within a segment selected from the group consisting of:
a segment of chromosome 1 bordered by D1S200 and D1S1631;
a segment of chromosome 5 bordered by D5S2848 and D5S589;
a segment of chromosome 6 bordered by D6S502 and D6S503;
a segment of chromosome 6 bordered by D6S1043 and D6S1007;
a segment of chromosome 7 bordered by D7S2195 and D7S559;
a segment of chromosome 7 bordered by D7S1808 and D7S2846;
a segment of chromosome 8 bordered by D8S1113 and D8S1132;
a segment of chromosome 9 bordered by D9S922 and D9S253;
a segment of chromosome 10 bordered by D10S1221 and D10S1432;
a segment of chromosome 11 bordered by D11S4131-qter;
a segment of chromosome 11 bordered by D11S912-qter;
a segment of chromosome 13 bordered by D13S1283 and D13S779;
a segment of chromosome 15 bordered by D15S822 and D15S643;
a segment of chromosome 17 bordered by D17S1298 and D17S787;
a segment of chromosome 17 bordered by D17S2196 and D17S1290;
a segment of chromosome 19 bordered by D19S1034 and D19S1165; and
a segment of chromosome 21 bordered by Pter-D21 S1442.

6. The method according to claim 5, wherein said determining the presence or absence of an allele of a polymorphic marker in the subject is performed utilizing DNA or RNA.

7. The method according to claim 5, wherein said method is a diagnostic method.

8. The method according to claim 5, wherein said method is a prognostic method.

9. The method according to claim 5, wherein said subject is human.

10. An oligonucleotide primer for amplification of an allele which is associated with asthma or Alzheimer's disease, wherein said allele is located at a locus in a region selected from the group consisting of:
a segment of chromosome 1 bordered by D1S200 and D1S11631;
a segment of chromosome 5 bordered by D5S2848 and D5S589;
a segment of chromosome 6 bordered by D6S502 and D6S503;
a segment of chromosome 6 bordered by D6S1043 and D6S1007;
a segment of chromosome 7 bordered by D7S2195 and D7S559;
a segment of chromosome 7 bordered by D7S1808 and D7S2846;
a segment of chromosome 8 bordered by D8S1113 and D8S1132;
a segment of chromosome 9 bordered by D9S922 and D9S253;
a segment of chromosome 10 bordered by D10S1221 and D10S1432;
a segment of chromosome 11 bordered by D11S4131-qter;
a segment of chromosome 11 bordered by D11S912-qter;
a segment of chromosome 13 bordered by D13S1283 and D13S779;
a segment of chromosome 15 bordered by D15S822 and D15S643;
a segment of chromosome 17 bordered by D17S1298 and D17S787;
a segment of chromosome 17 bordered by D17S2196 and D17S1290;
a segment of chromosome 19 bordered by D19S1034 and D19S1165; and
a segment of chromosome 21 bordered by Pter-D21S1442.

11. The oligonucleotide primer of claim 10, wherein said primer is from 5 to 50 nucleotides in length.

12. An assay for detecting a gene related to an asthma and/or atopy disorder comprising:
providing a biological sample comprising genomic DNA from a patient suspected of having or at risk for developing said asthma and/or atopy disorder;
using a probe directed toward to a region of a polymorphic marker in the subject, wherein (i) the marker is associated with a phenotypic marker of asthma and/or atopy, and wherein (ii) the polymorphic marker is within a segment selected from the group consisting of:
a segment of chromosome 1 bordered by D1S200 and D1S1631;
a segment of chromosome 5 bordered by D5S2848 and D5S589;
a segment of chromosome 6 bordered by D6S502 and D6S503;
a segment of chromosome 6 bordered by D6S1043 and D6S1007;
a segment of chromosome 7 bordered by D7S2195 and D7S559;
a segment of chromosome 7 bordered by D7S1808 and D7S2846;
a segment of chromosome 8 bordered by D8S1113 and D8S1132;
a segment of chromosome 9 bordered by D9S922 and D9S253;
a segment of chromosome 10 bordered by D10S1221 and D10S1432;
a segment of chromosome 11 bordered by D11S4131-qter;
a segment of chromosome 12 bordered by D11S912-qter;
a segment of chromosome 13 bordered by D13S1283 and D13S779;
a segment of chromosome 15 bordered by D15S822 and D15S643;
a segment of chromosome 17 bordered by D17S1298 and D17S787;
a segment of chromosome 17 bordered by D17S2196 and D17S1290;
a segment of chromosome 19 bordered by D19S1034 and D19S1165; and
a segment of chromosome 21 bordered by Pter-D21S1442; and
detecting duplications in the region of the genomic sequence of the group of chromosomes listed above.

13-14. (canceled)

15. The method of screening a subject for asthma and/or atopy according to claim 1 further comprising:
detecting the presence or absence of at least one or more markers linked to asthma and/or atopy, wherein the presence of said marker indicates that the subject is afflicted with or at risk of developing asthma and/or atopy, and wherein said marker is a segment of a chromosome bordered by D6S1043 and D6S1007 or within three centimorgans thereof.

16. The method according to claim 15, wherein said subject is of Greek descent.

17. The method of screening a subject for asthma and/or atopy according to claim 1 further comprising:
detecting the presence or absence of at least one or more markers linked to asthma and/or atopy, wherein the presence of said marker indicates that the subject is afflicted with or at risk of developing asthma and/or atopy, and wherein said marker is a segment of a chromosome bordered by D13S1283 and D13S779 or within three centimorgans thereof.

18. The method according to claim 17, wherein said subject is of Norwegian descent.

19-20. (canceled)

21. The method according to claim 5, wherein said subject is a Caucasian.

22. The method according to claim 1, wherein the marker is located in the ASTH_I/J region of chromosome 11.
